(19) United States
(12) Patent Application Publication
(10) Pub. No.: US 2024/0245743 A1
(43) Pub. Date: Jul. 25, 2024

(54) USE OF CINNAMOMUM ZEYLANICUM ESSENTIAL OIL (EO) AS HUMAN MELANOMA CELLS DEATH INDUCER AND AS POSSIBLE ADJUVANT IN PHARMACOLOGICAL THERAPIES

(71) Applicant: CONSIGLIO NAZIONALE DELLE RICERCHE, Roma (IT)

(72) Inventors: Francesca MARIANI, Roma (IT); Igea D'AGNANO, Roma (IT); Giulia CAPPELLI, Rome (IT); Cynthia CAPUTO, Roma (IT); Carlo PASTORE, Roma (IT); Giuseppe RUBERTO, Roma (IT)

(73) Assignee: CONSIGLIO NAZIONALE DELLE RICERCHE, Roma (IT)

(21) Appl. No.: 18/290,419

(22) PCT Filed: May 6, 2022
(86) PCT No.: PCT/IB2022/054196

§ 371 Date: Nov. 13, 2023

(30) Foreign Application Priority Data
May 11, 2021 (IT) 102021000012050

(51) Int. Cl. A61K 36/54; A61K 45/06; A61P 35/00

(52) U.S. Cl. CPC A61K 36/54; A61K 45/06; A61P 35/00

(57) ABSTRACT

The present invention relates to a di Cinnamomum zeylanicum EO, or to an association comprising said EO and one or more antitumour drug, for use in the treatment or in adjuvating the treatment of melanoma. The invention relates also to a pharmaceutical composition comprising said EO or said association and at least a pharmaceutically acceptable carrier for use in the treatment or in adjuvating the treatment of melanoma.

### BACKGROUND OF THE INVENTION

The present invention relates to a Cinnamomum zeylanicum essential oil (EO) which is particularly effective for use in the treatment of melanoma or in adjuvating the treatment of melanoma. The invention also relates to an association of said EO and one or more anti-tumour drug, as well as to a pharmaceutical composition comprising said EO or said association for use in the treatment of melanoma.

### STATE OF THE ART

The incidence of malignant melanoma, a neoplasm originating from melanocytes, is rapidly increasing all over the world. Although surgical excision still remains a definitive treatment for primary cutaneous melanoma, it is not applicable in cases of metastatic melanoma, which generally has a poor prognosis, with an average survival of 8-10 months and represents one of the major causes of morbidity and mortality. The poor prognosis of human melanoma is partly due to the limited therapeutic options available. Surgery and radiotherapy primarily provide palliative care and chemotherapy has failed to show any consistent survival benefit.

Although the therapeutic approach to the patient suffering from metastatic melanoma has shown significant progress in recent years thanks to the development of new pharmacological agents based on immune- and oncogen-targeted therapeutic strategies, the effect of these new therapeutic strategies on overall survival remains still variable today.

Therefore, in front of this scenario, the need to develop therapeutic approaches of greater clinical efficacy, which are able to significantly improve the survival of an increasingly higher proportion of patients with metastatic melanoma, is still evident. Cinnamons belong to different plant species that, although related, have very different components in their essential oils.

In particular, “Ceylon” and “Cassia” cinnamons powder and extracts are not obtained from the same plant species.

Cassia-type cinnamons include the following:
Cinnamomum burmannii, commonly called Indonesian Cinnamon, Indonesian Cassia, or Java Cinnamon
Cinnamomum cassia, also known as Cinnamomum aromaticum, and commonly called Chinese Cinnamon or Chinese Cassia
Cinnamomum loureiroi, commonly called Vietnamese Cinnamon, Vietnamese Cassia, Saigon Cinnamon, or Saigon Cassia.

Conversely, scientific names for Ceylon Cinnamon are Cinnamomum zeylanicum and Cinnamomum verum. The word “verum” in this species name comes from the Latin word verus for “true” which is the reason why this kind of cinnamon is also called “true cinnamon.”

In particular, what true cinnamon and cassia do not have in common is their Coumarin and Safrole content.

Coumarins are naturally occurring plant components that have strong anticoagulant properties.

These are other features, known in the art, that differentiate the two kinds of cinnamons.

| Cinnamomum cassia, from China, | Cinnamomun zeylanicum (“true”), |
|---|---|
| Vietnam or Java | from Sri Lanka |
| Toxic to liver if taken for prolonged | Generally safe |
| use | |
| Hard/rough in texture, not easily | Soft texture, easily broken |
| broken | |
| Dark brown and reddish in color | Light brown in color |
| Pungent and very spicy flavor | Soft and sweet aromatic |
| Native to China, India, Vietnam, | Native to Sri Lanka |
| Indonesia | |
| Only inward folded, empty cavity | Several folds of layer like a sigar |
| 0.45-5% Coumarin | <0.0005% Coumarin |
| 0.2% Safrole | <0.1% Safrole |

In C.cassia there are high level of Coumarin, about 1220 times more than C.zeylanicum. Coumarin is o hydroxycinnamic acid. Has anticoagulant activity by competing with Vitamin K. In large amounts, it can cause liver and kidney damage over time.

The state of the art reports various anti-cancer properties of Cinnamon extracts and of cinnamaldehyde, in most cases the state of the art refers to Cinnamomum cassia.

### SUMMARY OF THE INVENTION

The present invention provides a Cinnamomum zeylanicum (C. zeylanicum), or Ceylon Cinnamon, essential oil (EO) as an effective solution for the treatment or in adjuvating the treatment, of melanoma, in particular of metastatic melanoma, for which very few valid therapeutic approaches exist. The inventors have, in fact, discovered that a C. zeylanicum EO characterised by a specific composition of bioactive molecules, is capable of selectively blocking the replication of a cell line of human metastatic melanoma.

Surprisingly, not all C. zeylanicum essential oils show therapeutic activity in the treatment of melanoma and a precise combination of bioactive molecules is essential in order to obtain an effective therapeutic activity.

The oil of the invention, with the specific components amounts as defined in the description and in the claims, shows a cytotoxic activity specific for melanoma cancer cells and no cytotoxic activity on control cells.

By analysing the effect of different essential oils obtained by C. zeylanicum in the treatment of human metastatic melanoma cells, the inventors have found that a C. zeylanicum EO, characterised by a particular distinctive content of bioactive molecules, that is, by a higher content of E-cinnamaldehyde (50-58%), as well as, for example, by a reduced content of eugenol (3-8%) shows a greater anti-proliferative effect when compared to essential oils comprising the same bioactive molecules but in different quantities.

Cinnamaldehyde alone, did not show the properties of the oil of the invention. In addition, only C. zeylanicum EOs having the due amounts of classes of substances as defined in the description and in the claims showed a significant cytotoxic effect, herein defined as at least 45% reduction of cell viability on M14 melanoma cells (treated with 10 μg/ml EO for 24 hours), and in vivo in patients, whereas the other tested C. zeylanicum EOs did show a non-relevant cytotoxic effect, i.e. from 0 to 10% for EO5, EO3, EO6 or in any case considered insufficient, i.e. below 45% for EO2 and EO4 as shown in FIG. 1 and in unpublished data (EO3 and EO6).

In addition, the essential oils of the invention, being from C. zeylanicum and not from Cinnamomum cassia, due to their extremely low content of Coumarin (<0.0005%) and safrole (<0.1%, preferably <0.05%), do not exert the negative side effects related to these two compounds, that render C. cassia EOs unsuitable for treating oncologic patients, which are already undergoing invasive and extremely toxic therapies. The use in oncologic patients of an oil comprising higher amounts of compounds having relevant anticoagulant properties such as Coumarin (which in C. cassia EOs is normally from 0.45 to 5%) and Safrole, which is known in the art for showing a weak carcinogenicity in rats is, clearly, not desirable.

As well demonstrated by the experimental results, (FIGS. 1-10), the C. zeylanicum EO according to the formulation of the present invention, is capable of selectively increasing the oxidative stress, the damage to the mitochondrial membrane, as well as the intracellular ferrous iron (Fe²⁺) in melanoma cells without, on the other hand, damaging peripheral blood cells. In other words, the treatment of melanoma cells with the bioactive molecules of the C. zeylanicum EO as herein described and claimed, allows the increase of intracellular production of reactive oxygen species (ROS), thereby producing a pro-oxidative effect in the targeted melanoma cells. Said C. zeylanicum EO is also capable of regulating the quantity of ferrous iron (Fe²⁺) or metabolically active iron, or Labile Iron Pool (LIP), in melanoma cells and to modulate a group of genes that are responsible of intracellular iron metabolism. As evident from the experimental analysis, the results of which are reported in the figures and in the examples, the treatment with said C. zeylanicum EO is capable of depolarising the mitochondrial membrane, thereby damaging the energetic metabolism of melanoma cells. The technical consequence of this result is the inhibition of cellular growth of about 50% of the melanoma treated cells. The C. zeylanicum EO object of the present invention, has also shown the ability to improve the effect of an anti-tumour drug, Tamoxifen (TAM), belonging to the family of selective oestrogen receptor modulators, in the treatment of metastatic melanoma cells. As clearly described in the experimental section (FIGS. 4a and 4b), the administration of la C. zeylanicum EO in association with TAM, compared to the sole TAM administration, sensitizes the melanoma cells to the drug, in particular to a drug concentration to which the cells are normally resistant.

The experimental results depicted in FIG. 4e also demonstrate that the C. zeylanicum EO object of the present invention is capable of improving in a significative way the effect of Taxol (Paclitaxel), a known antimitotic drug, on melanoma cells. Where the administration of Taxol alone showed a very low induction of death in M14 cells (less than 10%), the administration of the EO object of the invention in combination with the drug increased cell death by more than 60% (p=0.0002).

As also highlighted by the experimental results reported in FIGS. 4g and 4h, the authors of the present invention have demonstrated that the EO object of the present invention is able to significantly enhance the effect of Dabrafenib (DAB), a well-known drug used in the treatment of patients with melanomas caused by the BRAF V600 mutation.

In light of the therapeutic effects described above, a therapeutic strategy based on the administration of C. zeylanicum EO in association with one or more anticancer drugs would allow to decrease the necessary drug doses (as demonstrated for Taxol and Dabrafenib), while providing a solution of greater efficacy than conventional therapeutic approaches in the treatment of metastatic melanoma.

Furthermore, the administration of the EO object of the invention in association with Tamoxifen, which significantly increases the cytotoxicity induced on M14 (TAM vs TAM+EO p<0.0001; TAM+EO vs EO p=0.0009), represents a possible alternative treatment option for patients without the BRAF V600 mutation, who therefore cannot be treated with Dabrafenib.

The percentages of the components of the oil of the invention, as reported in the description and in the claims, are intended with respect to the total volume of said oil. A first aspect of the invention hence relates to a C. zeylanicum essential oil for use in the treatment of melanoma or to adjuvate the treatment of melanoma, wherein said essential oil comprises:
E-cinnamaldehyde from 50 to 58%, and
eugenol from 3 to 8%;
to an association of said oil with one or more anticancer drugs in the treatment of melanoma and to a pharmaceutical composition comprising said EO or said association, and at least one pharmaceutically acceptable carrier for use in the treatment or in adjuvating the treatment of melanoma.

Other advantages and features of the present invention will become apparent from the following detailed description.

### BRIEF DESCRIPTION OF THE FIGURES

In all the experiments that have been carried out, the results of which are reported in FIGS. 1-10, the EOs indicated as EO7 and EO8 in table 1 have been used. The results obtained are comparable for the two oils used and the values shown are averages of the experiments carried out with both oils.

FIG. 1. a) Effect of the different EOs of C. zeylanicum (EO) tested on M14 melanoma cells to evaluate their effectiveness on cell viability (10 μg/ml for 24 hrs). The composition of the oils used is shown in table 1. b) Antiproliferative effect of C. zeylanicum EO on melanoma M14 cells: curves doses-response for C. zeylanicum EO (CINN EO) at the indicated EO concentrations (μg/ml). CTRL, untreated cells. The values represent the average of at least three experiments±the standard deviation. The arrow indicates the start of the treatment with the EO.

FIG. 2. Effect of the C. zeylanicum EO (CINN EO) on the distribution of M14 cells in the phases of the cell cycle. The cells were treated with the EO dose equal to 10 μg/ml for the indicated times. Untreated (CTRL) and treated cells were stained with Propidium Iodide to analyse the DNA content by flow cytometry. Using the ModFit program, the percentages of the cells in the different phases of the cell cycle were estimated: phase G0/G1 (light gray), phase S (black) and phase G2/M (dark gray). The shown histograms are representative of the analysis with ModFit having carried out at least 3 experiments with similar results for each condition. The number shown under each panel represents the treatment time in hours.

FIG. 3. a) Production of reactive oxygen species (ROS) in M14 cells and in human peripheral blood mononuclear cells (or human Peripheral Blood Mononuclear cells, hu-PBMCs) following the treatment with EO.
b) Mitochondrial membrane potential (mt-Δϕ) in the M14 cells and in PBMCs.
c) Cytograms representative of the expression of Puromycin analysed by flow cytometry. M14 cells were exposed to the 1 μM dose of Puromycin for 4 hours and then analysed by flow cytometry to evaluate their expression. NEG, negative control without primary antibody; PURO, sample with anti-Puromycin antibody.
d) Cytograms representative of the incorporation of Puromycin following the treatment of M14 cells with the C. zeylanicum EO. CTRL, untreated cells; CINN-EO, cells treated with EO.
e) Analysis of the mean fluorescence intensities (MFI) associated with the expression of Puromycin, derived from the cytograms in d), which indicate the amount of intracellular Iron (II) after treatment with C. zeylanicum EO. The values were normalised with respect to the total protein content in the cells. The reported values represent an average of three independent experiments.
f) Expression of the indicated genes in Monocyte-Derived Macrophages (or Monocyte-derived-Macrophages, MDMs) and in M14 following treatment with the EO.

FIG. 4. Effects on M14 cell survival of the combination of the C. zeylanicum EO with the drug Tamoxifen (TAM). a) Colony formation assay for M14 cells exposed to the C. zeylanicum EO used alone or in combination with TAM for 24 hours. Cell survival rates were calculated as the percentage of colonies formed in each sample compared to those formed in the untreated control sample. b) Representative images of the coloured colonies in each sample. c) Expression of the indicated genes following treatment with the C. zeylanicum EO±TAM. d) Comparison between the cytotoxic effect of cinnamaldehyde alone and the whole C. zeylanicum EO on M14 cells, by colony formation assay. e) Colony formation assay for M14 cells exposed to the C. zeylanicum EO used alone or in combination with Taxol (TAX) for 24 hours (p=0.0002). Left panel, representative images of the coloured colonies in each sample; right panel, statistical analysis of the 3 experiments conducted. f) Dose-response effect of Dabrafenib (DAB) used at the concentrations indicated in the figure on M14 at 24 hours of treatment (number of viable cells after treatment); g colony formation assays for M14 cells exposed to the C. zeylanicum EO used alone or in combination with the 1 nM and 1 μM concentrations of DAB. The statistical analysis of the 3 experiments conducted is shown; h) Representative images of the coloured colonies in each sample.

FIG. 5. Effects of the C. zeylanicum EO (CINN-EO) on the expression of the indicated proteins used individually or in combination with TAM by western blot analysis. Left panel, representative western blots showing the intensities of the expression bands of the proteins indicated in M14 cells treated with the C. zeylanicum EO±TAM; CTRL, untreated cells control. a) Expression of the HSP72-73 and Cyclin B1 proteins. b) Expression of the p53 protein. c) Expression of the HMOX1 protein. d) The graphs shown, show the values of increase or decrease in the expression of the individual proteins calculated on the basis of the densitometric measurement of the expression bands (as in a, b and c) obtained from the average of 3 similar experiments. The expression of the GAPDH protein was used in order to control the amount of proteins loaded in each blot.

FIG. 6. Analysis of the gene expression profile in normal melanocytes (HEMs, Human Epidermal Melanocytes), primary (Primary) and metastatic (Metastatic) melanoma tumour tissue from patients with melanoma included in public databases, such as The Cancer Genome Atlas (TCGA) and the Gene Expression Omnibus (GEO). Three different sets of gene expression data were obtained from TCGA and GEO, selecting data obtained with the same Affymetrix type gene-specific probes. The arrow shown in each panel indicates the tendency of the expression of each gene in M14 cells treated with the C. zeylanicum EO with respect to the untreated control. a) Genes whose expression is increased (upward arrow) in M14 cells exposed to the C. zeylanicum EO. b) Genes whose expression is repressed (downward arrow) in the M14 cells we treated with the EO and which are instead induced in primary (Primary) and metastatic (Metastatic) melanomas vs healthy melanocytes (HEMs).

FIG. 7. The Protoporphyrin IX inhibitor (SnPPIX), by blocking the HMOX1 protein, makes the M14 cells recover the inhibition of cell proliferation induced by the C. zeylanicum EO. I The data shown were obtained by means of a colorimetric cell proliferation assay (MTS) on M14 cells treated with the EO used alone or in combination with the SnPPIX inhibitor (5 μM) for 24 hours. Values shown are means±standard deviation of three independent experiments.

FIG. 8. Trend of LDH and S100, biomarkers of response to therapeutic treatment, and association with the relative % reduction of lesions at t1.

FIG. 9. Analysis of the statistical significance of the differences in LDH and S100 values. Box a) describes the trend of the two biomarkers in patients treated with conventional therapy plus OE. The differences between t0 and t1 (3/6 months of follow up) were significant (LDH p=0.01, S100 p=0.0066).

| LDH | | S100 | |
|---|---|---|---|
| How big is the difference? | | How big is the difference? | |
| Average of the | 252.5 | Average of the | 3.150 |
| differences | | differences | |
| 95% confidence | From | 95% confidence | From |
| interval | 109.2 to 395.8 | interval | 1.666 to 4.634 |
| R squared | 0.9129 | R squared | 0.9383 |
| How efficient is the pairing | | How efficient is the pairing | |
| (pairing)? | | (pairing)? | |
| Correlation | 0.9753 | Correlation | 0.9899 |
| coefficient | | coefficient | |
| P value (one tail) | 0.0124 | P value (one tail) | 0.0051 |
| Summary P value | * | Summary P value | ** |
| Pairing was | yes | Pairing was | yes |
| significatively | | significatively | |
| effective? | | effective? | |

Box b) describes the trend of the two biomarkers in patients treated with conventional therapy only. The differences between t0 and t1 (3/6 months of follow up) were only significant for S100, with a much lower p value (p=0.01 vs p=0.0066).

| LDH | | S100 | |
|---|---|---|---|
| How big is the difference? | | How big is the difference? | |
| Average of the | 81.33 | Average of the | 1.400 |
| differences | | differences | |
| 95% confidence | From | 95% confidence | From |
| interval | −54.33 to 217.0 | interval | 0.4683 to 2.332 |
| R squared | 0.3221 | R squared | 0.7490 |
| How efficient is the pairing | | How efficient is the pairing | |
| (pairing)? | | (pairing)? | |
| Correlation | 0.5909 | Correlation | 0.9420 |
| coefficient | | coefficient | |
| P value (one tail) | 0.1084 | P value (one tail) | 0.0025 |
| Summary P value | ns | Summary P value | ** |
| Pairing was | No | Pairing was | Si |
| significatively | | significatively | |
| effective? | | effective? | |

Box c) compares the variations in t1 vs t0 between the two groups. The differences between these ratios, although not statistically significant, show a clear trend towards a greater reduction in LDH during 3/6 months of treatment with EO added to conventional therapies.

| LDH | | S100 | |
|---|---|---|---|
| Unpaired T test (unpaired) | | Unpaired T test (unpaired) | |
| P value | 0.938 | P value | 0.2571 |
| P value summary | ns | P value summary | ns |
| F test for variances comparison | | F test for variances comparison | |
| F, DFn, Dfd | 15, 99, 5, 3 | F, DFn, Dfd | 3, 221, 5, 3 |
| P value | 0.0452 | P value | 0.3645 |
| Summary P value | * | Summary P value | ns |
| Are the variances | Si | Are the variances | No |
| significatively | | significatively | |
| different? | | different? | |

FIG. 10. a) Analysis of the statistical significance of the differences in the % reduction of metastatic lesions between the two groups of patients with metastatic melanoma (see table 2 and table 3). b) outline of the follow-up period.

Unpaired T test (unpaired)

| | |
|---|---|
| P value | 0.0351 |
| P value summary | * |
| Are the averages | Yes |
| significatively different? | |
| (P < 0.05) | |

How was pairing effective (pairing)?

| | |
|---|---|
| Correlation coefficient | 0.9553 |
| P value (one tail) | 0.0223 |
| Summary P value | * |
| Pairing was significatively | Yes |
| effective? | |

FIG. 11 shows the comparison of the metabolic composition (in % values) of the C. zeylanicum EO8 of the invention (table 1 below) with a published C. cassia bark EO (source: LI, Z. Q., LUO, L., HUANG, R. and XIA, Y. Q. (1998). Chemical studies of cinnamon, true plants from Yunnan province. Yunnan Daxue, Xuebao ziram kexueban. 20(Suppl): 337-379). The ratio between compounds in C. zeylanicum EO8 (Table 1 below) and C. cassia EO as described in Table. 11.7 of Li et al 1998 (table 4 below) is reported in the figure. As clear from the ratio C. zeylanicum:C. cassia, a first group of 16 compound resulted significantly more abundant in C. zeylanicum, while a second group of 18 compounds is more abundant in C. cassia, importantly including Coumarin and Safrole. Additionally, a third group of compounds present in C. cassia is totally absent in C. zeylanicum.

### GLOSSARY

The terms used in the present description, unless otherwise indicated, have the meaning commonly used in the state of the art.

As Essential Oils of Cinnamomum zeylanicum, or Ceylon Cinnamon (C. zeylanicum), present day Sri Lanka, also known as “essences”, natural compounds of vegetable origin consisting of a mixture of volatile substances at room temperature, generally having a terpenic nature, are intended. Said compounds can be present within different parts of plants, accumulated within their specialized structures, more or less on the surface. In some plants, for example, the essential oil is concentrated in the leaves, in other cases it is present in the bark, fruits, flowers, or even in the roots of the plant.

The term “melanoma” refers to a neoplasm that originates from the cells that produce melanin, called melanocytes. Melanoma can spread to other tissues and organs in the body, including the lungs, liver, or brain. In this case “metastatic melanoma” or “Stage IV” melanoma is used.

Ferrous iron, or labile iron, or 2+iron, or metabolically active iron (labile iron pool, or LIP) is the transition element Iron in its reduced state (the oxidized state is Fe³⁺). This ferrous iron participates in the Fenton reaction and contributes to the production of Reactive Oxygen Species (or ROS) in the cell.

The heme oxygenase protein (or HMOX1) is a microsomal enzyme that catalyzes the opening of the heme ring with the formation of biliverdin, iron and carbon monoxide (CO). Biliverdin is later converted into Bilirubin, which is an important antioxidant.

LDH, or Lactate dehydrogenase, is a key tetrameric enzyme in the glycolytic pathway. Catalyses the reversible conversion of pyruvate into lactate, coupled with the oxidation of Nicotinamide dinucleotide dehydrogenase (NADH) to NAD+ in the final step of the glycolytic pathway. An elevated LDH is well known as a negative prognostic biomarker in many tumours and is directly correlated with the increase in tumour growth, the index of proliferation, metastasis and survival of the tumour. LDH was included as a serological prognostic marker in stage IV metastatic melanoma according to the American Joint Committee Cancer classification.

S100 is a calcium-binding protein with a molecular weight of 21,000 found in the nervous system of vertebrates (particularly in astrocytes). In previous studies, the S100 protein was found in malignant melanoma tissue, where it was completely absent from normal skin samples and normal lymph nodes. Its use for the diagnosis of metastatic melanoma is recognized by the scientific community.

“Tamoxifen”: Tamoxifen or tamoxifen is an anticancer drug taken orally and belonging to the family of selective oestrogen receptor modulators. For twenty years it has been used in the treatment of advanced breast cancer. More recently it has been used as an adjuvant therapy, or in combination with a primary therapy. Its effectiveness has also been studied on other types of cancer, such as melanoma, uterine cancer, some leukaemia.

“Taxol (Paclitaxel)”: Taxol is a drug capable of inhibiting cellular mitosis. Until recently, the aim of treating locally advanced inoperable or metastatic melanoma could be considered almost exclusively palliative. The overall effectiveness of different antineoplastic strategies is rather limited, due to the high resistance to all forms of conventional treatments, including chemotherapy, radiotherapy and immunotherapy. Chemotherapy drugs, available for a long time (dacarbazine, fotemustine, temozolomide, platinum salts, paclitaxel) have shown a modest, non-curative effect in most cases. These drugs, although capable of inducing a tumour response both in monotherapy and in combination schemes of polychemotherapy, even in the case of association with immunotherapy, have never shown an improvement in survival. “BRAF inhibitors”: Most melanomas show activating mutations at the level of NRAS or

BRAF proto-oncogenes, components of the RAS-RAF-MEK-Erk (MAPK) signalling pathway. 40-60% of cutaneous melanomas express a mutation in BRAF. Mutations in the BRAF gene, which replace valine at the amino acid 600 position, result in constitutively activated BRAF proteins, which can result in constitutive activation of the downstream signal via the MAPK pathway and cell proliferation in the absence of growth factors, normally fundamental in cellular replication. The use of BRAF inhibitors and other downstream targets has shown preclinical and clinical benefit.

“Dabrafenib”: the combination of another BRAF inhibitor, GSK2118436 (dabrafenib) is currently under investigation. A phase III study comparing dabrafenib and dacarbazine was recently published. This study shows a median PFS (progression free survival) of 5.1 months for dabrafenib and 2.7 for dacarbazine. The ORR (objective response rate) was confirmed at 53% for dabrafenib and 19% for dacarbazine. This response rate is consistent with the result of a phase I study in which patients with advanced melanoma, untreated brain metastases, and other solid tumours achieved an ORR of 47%.

### DETAILED DESCRIPTION

As mentioned above, the authors of the present invention have found that the use of a C. zeylanicum EO characterized by a precise combination of bioactive molecules, represents a highly effective therapeutic solution for the treatment of metastatic melanoma or to assist said treatment. In particular, the C. zeylanicum EO identified by the inventors is characterised by a high content E-cinnamaldehyde (between the 50 and the 58% of the overall composition of the EO) and by a moderate Eugenol content (between about 3 and 8% of the total composition).

The surprising technical effect observed, as evident from the experimental results, is the ability of said C. zeylanicum EO to selectively inhibit the replication of metastatic human melanoma cells, leaving healthy cells unaltered, with greater efficacy than essential oils characterized by a different composition. Given that cinnamaldehyde is the most abundant compound in EO, the cytotoxic effect of cinnamaldehyde alone was analysed versus that induced by EO as a whole (FIG. 4.d). The experiment showed a significant increase in the cytotoxic effect of EO compared to that of cinnamaldehyde alone (the cell survival determined was 43.1% and 64.3%, respectively; p=0.02).

Therefore, a first aspect of the invention refers to C. zeylanicum EO for use in the treatment of melanoma or to adjuvate the treatment of melanoma, wherein said essential oil comprises:
E-cinnamaldehyde from 50 to 58%, and
eugenol from 3 to 8%.

According to an embodiment of the present invention, said C. zeylanicum EO comprises, in particular:
monoterpene hydrocarbons from 12 to 14.5%,
oxygenated monoterpenes from 13 to 14.5%,
sesquiterpenes from 9 to 11%,
E-cinnamaldehyde from 50 to 58%,
eugenol from 3 to 8%.

In particular, it is preferred that in all embodiments of the invention, coumarin is either absent or undetectable or in any case <0.0005% in the C. zeylanicum EO as described and claimed.

In addition, it is also preferred, that in all the embodiments of the invention safrole content is <0.1%, even more preferably <0.05% in C. zeylanicum EO as described and claimed. In an embodiment of the invention, the content of monoterpene hydrocarbons varies from 12 to 14.5% of said oil. In a possible embodiment, said monoterpene hydrocarbons comprise or consist of alpha-pinene (from 4.79 to 5.30%), beta-pinene (from 2 to 2.56%), alpha-Phellandrene (from 0.8 to 0.9%), p-cymene (from 0.6 to 0.9%) and limonene, (from 3.7 to 3.9%) each in the indicated percentages of said oil for a total of monoterpene hydrocarbons in the concentrations above.

In a further embodiment, the content of oxygenated monoterpenes is from 13.4 to 14.30% of said oil, preferably from 13.44% to 14.30% of said oil. In a possible embodiment, said oxygenated monoterpenes comprise or consist of 1.8 Cineol (from 2.72 to 3.06%) in Linalool (from 10.02 to 11.04%) in the indicated percentages of said oil, for a total of oxygenated monoterpenes in the concentrations indicated above.

In one embodiment, said essential oil has a content of monoterpene hydrocarbons and oxygenated monoterpenes in the ranges and/or components indicated above.

In a further embodiment, the content in sesquiterpenes is from 9.7 to 10.5% of said oil, preferably from 9.73% to 10.46% of said oil. In a possible embodiment said sesquiterpenes comprise or consist of beta-caryophyllene (from 8.66 to 9.44%), alpha-Copaene (from 0.19 to 0.28%), alpha-Humulene (from 0.21 to 0.54%), karyophyllene oxide (from 0.18 to 0.35%), each in the indicated percentages of said oil, for a total of sesquiterpenes in the concentrations indicated above.

According to an embodiment, said essential oil has a content in monoterpene hydrocarbons and in oxygenated monoterpenes and in sesquiterpenes in the ranges and/or in the components indicated above.

According to a further embodiment, applicable to all the embodiments indicated above, the content in E-cinnamaldehyde is from 50 to 57% of said oil, preferably from 50% to 56.55% of said oil, even more preferably from 50.55% to 56.55% of said oil.

According to another embodiment, applicable to all the embodiments indicated above, the eugenol content is from 3 to 7.5% of said oil, preferably from 3.5 to 7.5% of said oil, even more preferably from 3.74 to 7.03% of said oil.

The C. zeylanicum EO according to any of the previously described embodiments may further comprise:
o-methoxy cinnamaldehyde from 0.08 to 0.2% of said oil, preferably from 0.09 to 0.16% of said oil, and
E-cinnamyl acetate from 1.2 to 2.5% of said oil, preferably from 1.37 to 2.41% of said oil.

In a preferred embodiment, said essential oil has a content of monoterpene hydrocarbons from 12 to 14.5%, a content of oxygenated monoterpenes from 13.44 to 14.30%, a content of sesquiterpenes is from 9.73 to 10.46%, a content in E-cinnamaldehyde from 50.55% to 56.55% of said oil, a content in eugenol from 3.74 to 7.03% of said oil, a content in o-methoxy cinnamaldehyde from 0.09 to 0.16% of said oil. In a preferred embodiment said C. zeylanicum EO comprises:
monoterpene hydrocarbons from 12 to 14.02%,
oxygenated monoterpenes from 13.44 to 14.30%,
sesquiterpenes from 9.73 to 10.46%,
E-cinnamaldehyde from 50.55 to 56.55%,
eugenol from 3.74 to 7.03%,
o-methoxy cinnamaldehyde 0.09 to 0.16%
E-Cinnamyl acetate from 1.37 to 2.41%,

In a preferred embodiment, said monoterpene hydrocarbons comprise or consist of alpha-pinene, camphenene, sabinene, p-cymene and limonene; said oxygenated monoterpenes comprise or consist of 1-8 cineole and linalool; said sesquiterpenes comprise or consist of alpha-copaene, beta-caryophyllene, alpha-Humulene and Caryophyllene oxide.

Preferably, in all the embodiments of the present invention, an EO having a cytotoxic effect, defined as the reduction of cell viability of melanoma cells (such as M14 cells) of at least 45% when said cells are treated with 10 μg/ml of said EO for 24 hours, is selected.

The extraction of the C. zeylanicum essential oils object of the invention can be carried out using any of the techniques most commonly used in the field for the production of essential oils for therapeutic purposes, such as, for example, steam extraction or hydro distillation. According to an aspect of the present invention, the extraction of said C. zeylanicum EO is preferably carried out in a steam current.

As already mentioned, the selection of the starting plant material influences in an important way the precise biochemical composition of the essential oils, and consequently has a significant impact on their therapeutic effectiveness.

By way of example, essential oils extracted from the leaves of C. zeylanicum are characterized by a reduced content, in percentage, of cinnamic derivatives and by a higher content of eugenol. On the other hand, essential oils obtained from the bark of young plants of C. zeylanicum have a higher content of cinnamic compounds, such as E-cinnamaldehyde, and o-methoxy-cinnamaldehyde.

According to a preferred aspect of the present invention, said C. zeylanicum EO is exclusively extracted from the bark of the young branches of C. zeylanicum plants having an age greater than 2 years.

According to the invention, the C. zeylanicum EO at the aforementioned concentrations of the aforementioned components, can also be prepared by enriching the C. zeylanicum EO, if needed, in one or more of the classes of compounds indicated above in order to reach the ranges indicated above or indicated in the claims and/or by depleting the same EO in components whose concentrations are higher than those indicated in the present description and in the claims in order to obtain an EO with the desired requirements.

In particular, C. zeylanicum essential oils can also be enriched in one or more of E-cinnamaldehyde, o-methoxy cinnamaldehyde, and depleted in one or more of monoterpene, monoterpenes, sesquiterpenes and/or eugenol hydrocarbons in order to reach the ranges indicated above in any embodiment provided above.

A preferred embodiment according to the present invention relates to a C. zeylanicum EO for use in the treatment of melanoma or in adjuvating the treatment of melanoma, wherein said oil comprises:
14.02% of monoterpene hydrocarbons,
13.44% of oxygenated monoterpenes,
9.73% of sesquiterpenes,
56.55% of E-cinnamaldehyde,
3.74% of eugenol,
0.16% of o-methoxy cinnamaldehyde; and
1.37% of E-Cinnamyl acetate
or wherein said essential oil comprises:
14.02% of monoterpene hydrocarbons,
14.30% of oxygenated monoterpenes,
10.46% of sesquiterpenes,
50.55% of E-cinnamaldehyde,
7.03% of eugenol,
0.09% of o-methoxy cinnamaldehyde; And
2.41% of E-Cinnamyl acetate.

In one embodiment, the essential oil of the invention has the precise composition indicated in table 1 shown in this description, in the EO7 and/or EO8 column.

According to the present invention, the EO as defined in the present description and in the claims can be used for the therapeutic treatment or in adjuvating the therapeutic treatment of melanoma, in particular of primary melanomas or even of metastatic melanomas by administering the same. In one embodiment, therefore, the treatment can be carried out in association with other drugs, such as drugs conventionally used for the treatment of melanoma, such as for example anticancer drugs, at therapeutically effective dosages to a patient in need thereof.

By therapeutically effective dosages, dosages inhibiting the proliferation of cancer cells or reducing their number in said patient or in any case resulting in a reduction in lesions as assessed, for example, by computed tomography (CT) are intended.

The therapeutic efficacy in the patient undergoing treatment can be directly related to the decrease in the metabolic activity of melanoma cells, thanks to the pro-oxidant effect of the EO. In other words, the administration of a therapeutically effective amount of EO according to any of the embodiments described herein to a patient in need thereof, allows to obtain a reduction or inhibition of tumour cell growth, and/or a reduction or elimination of skin lesions in the patient.

According to an aspect of the invention, the essential oil according to any of the embodiments herein described can be administered, optionally in combination with one or more further active ingredients, such as for example anticancer drugs, to a patient in need thereof according to one or more administration method selected from topical, rectal, intramuscular, systemic, intravenous, or oral route

The administration of the EO according to the invention can be carried out in multiple unit doses that can be administered at appropriate time intervals, for example using the same or a different mode of administration.

Preferably, the essential oil as defined in the present description and in the claims can be administered orally and/or topically, in one or more daily doses. Preferably, the EO according to any of the embodiments herein described can be administered three times a day, after or in correspondence with the main meals.

According to an embodiment of the present invention, the EO according to any of the embodiments herein described can be administered orally according to a daily dosage between 50 and 200 mg/day, based on the indications of the EMA (10 May 2011 EMA/HMPC/706229/2009. Committee on Herbal Medicinal Products (HMPC) Community herbal monograph on Cinnamomum verum J. S. Presl, corticis aetheroleum). By way of example only, supposing to use an EO having a density equal to 1 g/mL and assuming that a drop of said EO corresponds to a volume approximately equal to 20 μL, said EO can be administered to a patient according to a dose equal to at 1-10 drops/day, preferably 10 drops/day (in this case 50 drops of EO will contain approximately 1 g of EO, i.e. 1000 mg of EO, therefore one drop will contain approximately 20 mg of EO). In cutaneous melanoma, the essential oil according to the invention can preferably be used topically, optionally also in combination with a systemic or oral administration, as such or in appropriate formulations such as creams, gels, emulsions.

According to a preferred aspect of the invention, the EO as described in the present description and in the claims can be administered topically inside a base cream.

A base cream suitable to be used for a topical application of the EO according to the invention is, for example, a hydrophilic base cream, that is a neutral oil-in-water emulsion, suitable as a carrier of the EO as such to facilitate the diffusion of active ingredients present therein on the skin.

According to one aspect of the invention, one or more aliquots of the EO according to the invention can be dissolved within a base cream in order to provide a therapeutically effective dosage for the treatment of skin melanoma. One or more drops of the EO according to the invention can for example be dissolved in a base cream in order to obtain a dilution of the EO as such at least of 1:100.

By way of example only, 10 50 μL drops of the essential oil according to the invention can be dissolved in 50 mL of base cream so as to obtain a dilution of the OE as such of 1:100.

The cream comprising the EO as such according to the invention can be applied directly to the affected area at the skin level, preferably twice a day.

According to one aspect of the invention, an appropriate dose of EO as such can also be added to a food, preferably a soft food, such as yogurt, to facilitate its intake.

In a preferred embodiment of the invention, two drops of the EO as such according to the invention will be dissolved in 100 g of yogurt, and preferably the yogurt thus obtained will be administered three times a day, preferably during or after the main meals.

The administration of the further optional active ingredients can take place simultaneously or in sequence and can take place by means of administration different from those used for said essential oil.

As already mentioned, the association of the C. zeylanicum EO object of the present invention and of one or more anticancer drugs, has an enormous therapeutic potential for the treatment of melanoma, thanks to the ability of said EO to favour an improvement in the effect of the drug and at the same time reducing the drug doses required for effective treatment.

In light of this technical effect, an aspect of the present invention therefore relates to an association of C. zeylanicum EO according to any of the embodiments herein described, and of one or more anticancer drugs for use in the treatment of melanoma or in adjuvating the treatment of melanoma.

According to an aspect of the invention, the EO according to any of the embodiments herein described, can therefore be administered in association with one or more anticancer drugs to a patient in need thereof.

The expression “in association”, used in the context of the present description, includes the possibility that said EO is administered concomitantly, i.e. simultaneously, with said one or more anticancer drugs, or in sequence, i.e. separately, with respect to the administration of said one or more anticancer drugs.

The term “concomitantly” or “simultaneously” is used in the context of this description to indicate an administration of EO carried out at the same time, for example through the same mode of administration, or within a short time interval with respect to the administration of said one or more anticancer drugs, using the same or a different mode of administration.

The expression “concomitantly” or “simultaneously” also means an administration of said EO carried out in a previous interval of time, provided it is close, to the administration of said one or more anticancer drugs.

By “close time interval” it is intended, for example, a time interval of 10 minutes, 20 minutes, 30 minutes, 50 minutes, 1 hour, starting from the administration of said EO or said one or more tumour drugs.

In the context of the present description, the expression “in sequence” is used to indicate an administration of the EO carried out separately over time with respect to the administration of said one or more anticancer drugs, i.e. before or after the administration of said one or more anticancer drugs. According to an aspect of the invention, the administration of said EO can be carried out from 2 to 48 hours before or after the administration of said one or more anticancer drugs, for example 3 hours, 4 hours, 6 hours, 10 hours, 12 hours, 16 hours, 18 hours, 24 hours, 36 hours, 48 hours before or after said administration. Sequential administration can also be carried out using the same method of administration for the EO and one or more anticancer drugs, or by using one or more different administration methods.

According to an embodiment, said one or more anticancer drugs can, therefore, be administered to a patient in need thereof according to one or more administration mode different from those used for said essential oil. In other words, the mode of administration of said one or more anticancer drugs, whether it is carried out concomitantly or in sequence with respect to the administration of the EO according to any of the embodiments of the invention, can be selected independently with respect to the mode of administration of said EO.

In particular, the most suitable mode of administration of said one or more anticancer drugs to said patient can be selected from any of the administration modes known in the field, and preferably among the administration modes commonly used for said one or more drugs in the conventional melanoma treatment therapies. Based on the chemical-physical characteristics of the anticancer drug or drugs selected for administration in association with the EO according to any of the forms described herein in the treatment of melanoma, a person skilled in the art will be able to select the suitable mode of administration as well as the appropriate dosages.

According to a preferred aspect of the invention, when administered in association with one or more anticancer drugs, the EO according to any one of the embodiments described herein can be administered topically and by one or more routes selected from the rectal, intramuscular, systemic, intravenous, or oral route. In addition, according to any form of administration described here, the EO can be additionally administered topically, for example: oral administration of the EO in association with one or more anticancer drugs and additional topical administration of the EO.

Examples of anticancer drugs that can be used in combination with said C. zeylanicum EO according to the present invention, can be drugs having a molecular target already known for the treatment of melanoma, such as antitumour inhibitors of MEK, inhibitors of mutated or non-mutated BRAF, or c-KIT inhibitors, as well as mitotic inhibitor drugs. In a particular embodiment, the combination with selective oestrogen receptor modulating drugs, such as, for example, Tamoxifen is excluded.

Non-limiting examples of MEK-inhibiting anticancer drugs include trametinib, cobimetinib, binimetinib; examples of mutated or unmutated BRAF antitumor inhibitors include vemurafenib, dabrafenib, encorafenib; examples of c-KIT inhibitors include imatinib and nilotinib; finally, examples of antimitotic inhibitors include mebendazole, vincristine, vinblastine, paclitaxel (Taxol), and docetaxel.

A preferred embodiment according to the present invention relates in particular to a combination of EO according to any of the embodiments described herein and dabrafenib for use in the treatment of melanoma.

A second preferred embodiment according to the present invention relates in particular to an association of EO according to any one of the embodiments described here and paclitaxel for use in the treatment of melanoma.

A further embodiment according to the invention relates to an association of EO according to any of the embodiments described herein and Tamoxifen for use in the treatment of melanoma.

According to an aspect of the invention, said one or more anticancer drugs can be administered according to a standard dosage, i.e. according to the dosage foreseen for said one or more drugs in conventional melanoma treatment therapies, while the EO according to any of the forms described herein, can be administered according to any of the dosages and/or dosage regimens defined in the present description.

Paclitaxel can be preferably administered by injection into a vein through a cannula, a thin tube that is introduced into a vein in the arm or hand; or by infusion into a vein (by drip administration) through a cannula, through the central venous catheter which is inserted under the skin in a vein near the collarbone, or through the so-called “PICC” line, acronym from the English “peripherally inserted central catheter”, which is inserted into a peripheral vein, usually in the arm.

Dabrafenib and/or Tamoxifen can preferably be administered orally, for example in capsule or tablet form.

The setting of the optimal dosage of said one or more anticancer drugs and/or of said EO may vary according to the mode of administration selected as well as according to the clinical picture of the patient undergoing the treatment (for example also based on age, weight body, sex of the patient) or, in particular, based on the severity or stage of progress of the disease.

In a preferred embodiment, said one or more anticancer drugs are administered at a lower dosage or for a lower number of treatment cycles than that commonly used in the therapy of melanoma.

The present invention further relates to a pharmaceutical composition for use in the treatment of melanoma, comprising the Cinnamomum zeylanicum essential oil of as defined above or in the claims, or to an association in any of the embodiments defined above, and at least one pharmaceutically acceptable excipient.

Excipient components suitable for use in the present invention, can be selected from those normally known in the state of the art such as, for example, stabilizers, preservatives, solvents, pH regulators, isotonic regulators, chelating agents, cryoprotective agents, diluting agents, binding agents, antioxidants, surfactants.

The composition according to the present invention can further include protective compounds, which can facilitate the specific transport and/or release of said EO and/or of said anticancer drug in the cells of interest. Such compounds can include any pharmacological transport system known in the art, for example biocompatible polymers, microparticle systems, liposomes, nanostructured materials, photosensitive capsules, nanoparticles, cationic lipids.

According to an aspect of the present invention, said composition preferably comprises lipid nanoparticles suitable for the delivery of said EO by oral administration. Advantageously, the conveyance with lipid nano particles enables to significantly reduce the gastric toxicity for oral intake of the composition.

According to an embodiment suitable for the treatment of patients affected by melanoma, the pharmaceutical composition of the present invention comprises said C. zeylanicum EO, in an amount comprised between 50 and 200 mg with respect to the total weight of the composition.

The composition can be divided into unit doses of about 65-70 mg of EO for a maximum daily dosage of about 200 mg/day.

The composition object of the invention, according to any of the embodiments herein described, can be administered according to any of the previously mentioned administration modes.

In a preferred embodiment of the present invention, said composition comprises said C. zeylanicum EO in an amount ranging from 50 to 200 mg with respect to the total weight of the composition, and one or more anticancer drugs in a concentration commonly used in therapy to depending on the drug selected or the drug cocktail selected, or even at concentrations lower than the total weight of said composition. The compositions according to any of the embodiments provided in the present description can be formulated in solid form, for example in the form of a hard capsule or soft-gel, liposomes, or in liquid form, such as, for example, suspension, solution, emulsion, syrup, spray. In a preferred embodiment, the oil object of the invention is administered as such, either directly by topic route or by adding it before use to the selected carrier.

The essential oil, or association, or composition formulated, according to any of the embodiments of the invention, may be administered in one or more doses at appropriate time intervals. According to an aspect of the invention, said EO, or association, or composition according to the invention will preferably be administered in three daily unit doses, for example at, before or after the main meals. The treatment can be continued until the patient no longer benefits from it or until complete recovery.

In particular, in case an EO according to the invention is used in combination with dabrafenib, it is preferable that, by way of example in the case of sequential administration, said association or composition, or dabrafenib alone, is administered at least one hour before or two hours after a meal due to possible side effects of food on the absorption of dabrafenib.

According to an aspect of the present invention, the overall duration of the treatment will be evaluated by the attending physician based on the patient's responses.

It is declared that the examples below have been carried out on cells available in cellular databases or on cells taken from informed patients who have given their express, free and informed consent to said collection and use on the basis of the current legislation.

### EXAMPLES

Some non-limiting examples of the compositions according to the present invention are provided below.

Selection of the C. zeylanicum Essential Oils

Subject of this study are six essential oils obtained from C. zeylanicum (EO1-EO8) indicated in Table 1, of which the first six are commercial samples and two are extracted directly from the plant material (100 g of cinnamon bark) by hydro distillation in an apparatus Clevenger-type for about 3 hours. Each EO sample was dried over anhydrous sodium sulphate and stored in a nitrogen atmosphere inside a sealed vial until use. The yields of both hydro distillation processes were 1.5% (v/w). Each analysis was repeated in triplicate. All essential oils have been stabilized in ethanol, in a ratio of 1:10 of EO and 98% pure ethanol and stored at −20° C. until use.

Gas-Chromatographic Analysis (GC) and GC-MS C. zeylanicum Essential Oils

A qualitative-quantitative analysis of C. zeylanicum EO1-EO8 samples under study was carried out by Gas Chromatography (GC), using a Shimadzu gas chromatograph, Model 17-A, coupled to a flame ionization detector (FID), and Class VP Chromatography Date System software version 4.3 (Shimadzu). An SPB-5 capillary column (15 m y 0.10 mm×0.15 um) was used, and helium was used as carrier gas (1 mL/min). The sample injection was carried out in “split mode” (1:200), and a volume equal to 1 μL of essential oil (4% of essential oil in CH2Cl2 v/v) was injected, using a temperature of 250° C. and 280° C., respectively for the injector and for the detector. The linear speed in the column is 19 cm/sec.

The oven temperature was maintained at 60° C. for one minute, and subsequently increased from 60 to 280° C. at a rate of 10° C. per minute, and finally maintained at 280° C. for one minute. The percentages of EO components were determined from the peak areas in the GC-FID profiles. The analysis by gas chromatography-mass spectrometry (GC-MS) was carried out in “fast mode” using a Shimadzu Gas chromatograph-MS (mod. GCMS-QP5050A), using the same column and experimental conditions used for the GC-FID, and a GCMS solution software, version 1.02 (Shimadzu). The ionization voltage is 70 eV, the electronic multiplier 900 V, the temperature of the ion source 180° C. The mass spectra were obtained in “scan mode” in the range of z/m between 40-400. The same EO solutions (1 μL) were injected in “split mode” (1:96).

The results of the analysis carried out are shown in Table 1 below

TABLE 1

| | RI | RI | | EO1 | EO2 | EO3 | EO4 | EO5 | EO6 | EO7 | EO8 |
|---|---|---|---|---|---|---|---|---|---|---|---|
| # | Lit. | Exp. | Class/Compound | % | | | | | | | |
| | | | Monoterpene hydrocarbons (16) | 7.48 | 8.36 | 0.23 | 11.07 | 9.95 | 3.26 | 14.02 | 14.02 |
| 3 | 927 | 926 | α-Thujene | 0.17 | 0.20 | | | 0.25 | 0.07 | 0.14 | 0.15 |
| 4 | 939 | 933 | α-Pinene | 1.16 | 1.34 | 0.08 | 1.48 | 1.07 | 0.74 | 5.30 | 4.79 |
| 5 | 954 | 948 | Camphene | 0.44 | 0.49 | 0.05 | 0.59 | 0.41 | 0.26 | 0.39 | 0.39 |
| 7 | 975 | 972 | Sabinene | 0.01 | 0.02 | 0.03 | 0.03 | 0.08 | | 0.03 | 0.05 |
| 8 | 979 | 975 | β-Pinene | 0.43 | 0.49 | | 0.38 | 0.36 | 0.26 | 2.51 | 2.64 |
| 9 | 991 | 987 | Myrcene | 0.09 | 0.12 | | 0.15 | 0.10 | | 0.15 | 0.15 |
| 10 | 1003 | 1000 | α-Phellandrene | 1.26 | 1.48 | | 1.30 | 0.91 | 0.24 | 0.84 | 0.80 |
| 11 | 1009 | 1015 | Δ-3-carene | 0.11 | 0.12 | | | | | 0.08 | 0.08 |
| 12 | 1017 | 1014 | α-Terpinene | 0.29 | 0.28 | | 1.07 | 0.93 | 0.06 | 0.09 | 0.13 |
| 13 | 1025 | 1022 | p-Cymene | 1.85 | 1.88 | 0.02 | 1.26 | 1.18 | 1.06 | 0.65 | 0.85 |
| 14 | 1029 | 1029 | Limonene | | | 0.05 | | | 0.56 | 3.72 | 3.86 |
| 15 | 1030 | 1028 | β-Phellandrene | 1.53 | 1.77 | | 4.34 | 4.26 | | | |
| 18 | 1037 | 1036 | β-Z-Ocimene | 0.01 | 0.04 | | 0.06 | 0.06 | | 0.02 | 0.02 |
| 20 | 1050 | 1047 | β-E-Ocimene | 0.03 | 0.03 | | 0.04 | 0.04 | | | |
| 21 | 1060 | 1058 | γ-Terpinene | | | | 0.19 | 0.18 | | 0.02 | |
| 24 | 1088 | 1076 | p-Mentha-2,4(8)-diene | | | | | | | 0.06 | 0.07 |
| 25 | 1089 | 1086 | Terpinolene | 0.10 | 0.12 | | 0.16 | 0.13 | | 0.03 | 0.04 |
| | | | Oxygenated monoterpenes (14) | 4.95 | 5.22 | 0.13 | 3.62 | 6.75 | 3.10 | 13.44 | 14.30 |
| 16 | 1031 | 1031 | 1,8-Cineole | 0.15 | 0.20 | | 0.08 | 1.05 | 0.12 | 3.06 | 2.72 |
| 23 | 1086 | 1064 | trans-Linalool oxide | | | | | | | 0.02 | 0.03 |
| 26 | 1097 | 1097 | Linalool | 4.18 | 4.38 | | 1.29 | 3.82 | 2.64 | 10.02 | 11.04 |
| 29 | 1121 | 1121 | dehydro-Sabine Ketone | | | | 0.15 | | | | |
| 30 | 1122 | 1122 | cis-p-Menth-2-en-1-ol | | | | 0.11 | 0.13 | | | |
| 31 | 1141 | 1141 | trans-p-Menth-2-en-1-ol | | | | | 0.09 | | | |
| 32 | 1146 | 1157 | Camphor | 0.01 | 0.03 | | | | | | |
| 34 | 1169 | 1168 | Borneol | 0.04 | 0.03 | 0.06 | 0.06 | 0.05 | | 0.03 | 0.04 |
| 35 | 1177 | 1177 | Terpinen-4-ol | 0.19 | 0.17 | | 1.27 | 0.53 | 0.07 | 0.09 | 0.24 |
| 36 | 1183 | 1191 | p-Cymen-8-ol | 0.03 | 0.04 | | | | | | |
| 37 | 1189 | 1190 | α-Terpineol | 0.36 | 0.37 | | 0.48 | 0.83 | 0.26 | 0.23 | 0.24 |
| 39 | 1210 | 1201 | cis-Sabinol | | | | | 0.07 | | | |
| 40 | 1221 | 1207 | cis-Sabinene hydrate | | | | 0.06 | 0.05 | | | |
| 48 | 1291 | 1288 | trans-Sabinyl acetate | | | 0.07 | | | | | |
| 51 | 1290 | 1294 | Thymol | | | | 0.09 | 0.08 | | | |
| 52 | 1299 | 1302 | Carvacrol | | | | 0.02 | 0.05 | | | |
| | | | Sesquiterpenes (31) | 4.87 | 4.80 | 0.75 | 3.36 | 3.60 | 5.74 | 9.73 | 10.46 |
| 55 | 1351 | 1323 | α-Cubebene | | | | | | | 0.07 | 0.03 |
| 58 | 1377 | 1379 | α-Copaene | | | 0.15 | 0.11 | 0.13 | 0.61 | 0.19 | 0.28 |
| 60 | 1387 | 1413 | β-Cubebene | 0.05 | | | | | | | |
| 62 | 1408 | 1426 | iso-Caryophyllene | 0.01 | 0.03 | | | | | | 0.04 |
| 63 | 1419 | 1425 | β-Caryophyllene | 3.99 | 3.98 | 0.07 | 2.27 | 2.00 | 2.25 | 8.66 | 9.44 |
| 64 | 1420 | 1444 | β-Ylangene | | | | | | | 0.04 | 0.05 |
| 66 | 1460 | 1455 | allo-Aromadendrene | | | 0.03 | | | | | |
| 67 | 1455 | 1461 | α-Humulene | 0.65 | 0.65 | | 0.42 | 0.34 | 0.39 | 0.54 | 0.21 |
| 68 | 1481 | 1477 | γ-Muurolene | | | 0.05 | | | | | |
| 69 | 1481 | 1486 | ar-Curcumene | 0.02 | | 0.02 | 0.03 | | | | |
| 70 | 1493 | 1505 | Guajene | 0.03 | 0.04 | | | | | | |
| 71 | 1498 | 1490 | α-Selinene | | | | | 0.04 | | | |
| 72 | 1500 | 1501 | α-Muurolene | | | 0.02 | 0.04 | 0.06 | | | |
| 73 | 1506 | 1502 | β-Bisabolene | | | 0.03 | | 0.07 | | | |
| 74 | 1496 | 1515 | Viridiflorene | 0.01 | | | | | | | |
| 75 | 1516 | 1518 | β-Curcumene | | | 0.04 | | | | | |
| 77 | 1523 | 1527 | Δ-Cadinene | 0.12 | 0.10 | 0.06 | | 0.08 | | | |
| 79 | 1546 | 1550 | α-Calacorene | | | 0.02 | | | | 0.02 | 0.02 |
| 80 | 1572 | 1562 | Caryophyllene alcohol | | | | 0.02 | 0.25 | | 0.02 | 0.02 |
| 81 | 1563 | 1570 | E-Nerolidol | | | 0.06 | | | | 0.02 | 0.02 |
| 82 | 1578 | 1581 | Spathulenol | | | 0.09 | 0.08 | 0.06 | 0.09 | | |
| 83 | 1583 | 1594 | Caryophyllene oxide | | | | 0.32 | 0.30 | 1.49 | 0.18 | 0.35 |
| 84 | 1585 | 1595 | Globulol | | | 0.08 | | | | | |
| 86 | 1608 | 1613 | Humulene epoxide | | | | | | 0.24 | | |
| 87 | 1629 | 1615 | 1-epi-Cubenol | | | | | 0.04 | | | |
| 88 | 1640 | 1648 | epi-α-Cadinol | | | | | 0.05 | | | |
| 89 | 1646 | 1652 | α-Muurolol | | | | | 0.03 | | | |
| 90 | 1660 | 1662 | Intermedeol | | | | | 0.06 | | | |
| 91 | 1670 | 1670 | 14-hydroxy-9-epi-E-Caryophyllene | | | | 0.09 | 0.05 | 0.17 | | |
| 92 | 1682 | 1677 | Occidentalol acetate | | | | | 0.04 | 0.52 | | |
| 93 | 1686 | 1690 | α-Bisabolene | | | 0.02 | | | | | |
| | | | Diterpenes (1) | | | | 0.20 | | | | |
| 96 | 1974 | 1977 | Sclarene | | | | 0.20 | | | | |
| | | | Others (31) | 80.85 | 80.17 | 97.51 | 80.79 | 79.15 | 85.63 | 62.62 | 60.82 |
| 1 | 877 | 877 | Styrene | | | 0.06 | | | | 0.02 | 0.02 |
| 2 | 931 | 925 | Cumene | | | | 0.24 | | | | |
| 6 | 960 | 960 | Benzaldehyde | 0.31 | 0.36 | 0.48 | 0.23 | 0.16 | 0.15 | 0.10 | 0.14 |
| 17 | 1025 | 1025 | Isobuthyl 2-methylbutanoate | | | | | | | 0.02 | 0.02 |
| 19 | 1045 | 1041 | Salicylaldehyde | | | 0.08 | | | | | |
| 22 | 1065 | 1072 | Acetophenone | 0.01 | 0.01 | 0.02 | | | | | |
| 27 | 1100 | 1100 | Isopenty1-2-methyl butanoate | | | | | 0.04 | | | |
| 28 | 1107 | 1107 | Phenylethyl alcohol | | | 0.27 | 0.02 | | | | |
| 33 | 1155 | 1161 | Hydrocinnamaldehyde | | | 0.37 | 0.38 | 0.46 | | 0.03 | 0.03 |
| 38 | 1196 | 1195 | Estragole | | | | 0.14 | | | | |
| 41 | 1219 | 1218 | Z-Cinnamaldehyde | 0.22 | 0.25 | 0.45 | 0.51 | 0.49 | 0.07 | 0.02 | 0.02 |
| 42 | 1227 | 1233 | Hydrocinnamyl alcohol | 0.01 | 0.02 | 0.05 | 0.03 | | | | |
| 43 | 1242 | 1245 | o-Anisaldehyde | | | 0.37 | | | | | |
| 44 | 1258 | 1252 | Phenyl ethyl acetate | | | 0.03 | | | | | |
| 45 | 1250 | 1260 | Chavicol | | | | | | 0.10 | | |
| 46 | 1261 | 1257 | Cinnamyl acetate | | | | 0.02 | | | | |
| 47 | 1270 | 1285 | E-Cinnamaldehyde | 55.26 | 53.04 | 87.04 | 66.25 | 65.82 | 1.31 | 56.55 | 50.55 |
| 49 | 1287 | 1293 | Safrole | 0.03 | 0.54 | | | | 0.96 | | |
| 50 | 1285 | 1290 | E-Anethole | 0.07 | 0.03 | | 0.03 | | | | |
| 53 | 1303 | 1306 | E-Cinnamyl alcohol | | | | 0.17 | 0.08 | 0.10 | | |
| 54 | 1319 | 1356 | α-methyl Cinnamaldehyde | 0.01 | 0.05 | | | | | | |
| 56 | 1359 | 1361 | Eugenol | 21.35 | 21.69 | 0.17 | 1.80 | 6.78 | 75.77 | 3.74 | 7.03 |
| 57 | 1368 | 1371 | Hydrocinnamyl acetate | 0.02 | 0.06 | | 0.13 | 0.08 | 0.08 | 0.03 | 0.04 |
| 59 | 1404 | 1403 | Methyl eugenol | 0.01 | 0.50 | | 0.02 | | | | |
| 61 | 1394 | 1419 | Vanillin | 0.04 | 0.02 | | | | | | |
| 65 | 1446 | 1451 | E-Cinnamyl acetate | 1.36 | 1.65 | 0.96 | 8.43 | 3.25 | 1.66 | 1.37 | 2.41 |
| 76 | 1523 | 1527 | Eugenol acetate | 0.77 | 0.78 | | | | 2.13 | | |
| 78 | 1529 | 1541 | o-methoxy Cinnamaldehyde | 0.03 | 0.03 | 7.13 | 0.52 | 0.30 | | 0.16 | 0.09 |
| 85 | 1613 | 1609 | Tetradecanal | 0.29 | 0.11 | | 0.02 | 0.07 | | | |
| 94 | 1760 | 1779 | Benzyl benzoate | 1.06 | 1.04 | 0.04 | 1.75 | 1.63 | 3.30 | 0.59 | 0.47 |
| 95 | 1860 | 1870 | Phenethyl benzoate | | | | 0.08 | | | | |
| | | | Monoterpene hydrocarbons | 7.48 | 8.36 | 0.23 | 11.07 | 9.95 | 3.26 | 14.02 | 14.02 |
| | | | Oxygenated monoterpenes | 4.95 | 5.22 | 0.13 | 3.62 | 6.75 | 3.10 | 13.44 | 14.30 |
| | | | Sesquiterpenes | 4.87 | 4.80 | 0.75 | 3.36 | 3.60 | 5.74 | 9.73 | 10.46 |
| | | | Diterpenes | | | | 0.20 | | | | |
| | | | Others | 80.85 | 80.17 | 97.51 | 80.79 | 79.15 | 85.63 | 62.62 | 60.82 |
| | | | Total | 98.16 | 98.55 | 98.61 | 99.04 | 99.46 | 97.74 | 99.81 | 99.60 |

The numbering #refers to the elution order, and the values (relative peak area) represent the averages of 3 determinations; RI Lit. indicates the Literature Retention Index (RI); RI Exp. Indicates the Retention Index relating to the standard mixture of n-alkanes on the SPB-5 column.

In all EOs of table 1 Cumarin content was below 0.0005%.

For all the cells of table 1 wherein no values are reported, the compound to which they refer is absent or in any case at a concentration below 0.01%.

The identity of the components of the different EOs was attributed on the basis of the GC retention indexes of each compound (relative to the C9-C20 n-alkanes on the SPB-5 column), on the basis of the computer correspondence of the MS spectral data with those of the MS NIST libraries, on the basis of the comparison between the fragmentation models with those reported in the literature and, when possible, on the basis of co-injections carried out with reference samples.

All subsequent experiments were carried out with the EOs indicated as EO7 and EO8 in Table 1, and human melanoma cells were used.

Culture of M14 Cells and Treatment with C. zeylanicum EO

Metastatic human melanoma M14 cells (also known as M14-MEL, UCLA-SO-M14, UCLA SO M14, UCLA-SO-14, Melanoma 14, M-14), derived from amelanotic metastatic melanoma cells (from a of vitiligo area) from a 33-year-old man, from the subcutaneous layer of the right buttock, were cultured in RPMI-1640 medium supplemented with 10% foetal bovine serum (Euroclone), 2 mM L-glutamine and 1% penicillin/streptomycin in a fully humidified incubator containing 5% CO₂ at 37° C. This line of human metastatic melanoma, purchased from the American Type Culture Collection (ATCC, https://www.lgcstandards-atcc.org) was genetically validated before conducting all the experiments, as per the certificate held by the authors.

M14 cells were treated with Cinnamomum zeylanicum (C. zeylanicum) Essential Oils (EO) according to three different doses (0.1, 1 and 10 μg/ml). The cytotoxicity of the different C. zeylanicum EOs was assessed by dose-response growth curves of the treated M14 cells. Viable cell counts (by excluding Trypan blue) were carried out daily for 3 days (72 hours, 48 hours of treatment) according to the scheme illustrated in the scheme below.

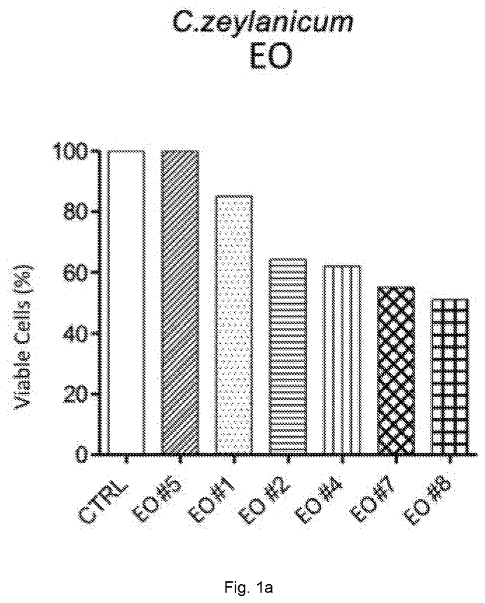

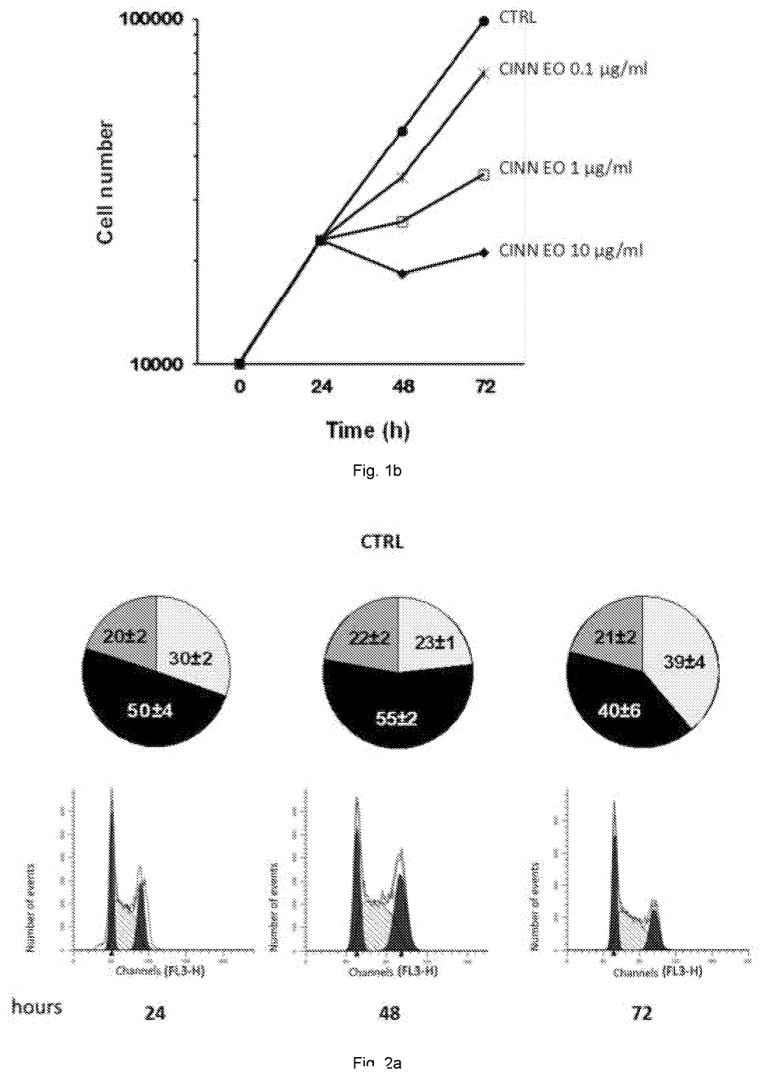

EO7 and EO8 showed the highest cytotoxic effect (45% and 49% respectively), compared to the other six EOs tested when used at 10 μg/ml on M14 cells for 24 hours (see FIG. 1.a), and therefore all subsequent experiments were carried out with EO7 and EO8.

As indicated in FIG. 1b, the use of increasing doses of EO induces increasing percentages of replication inhibition, measured at 24 (48 hours from t0) and 48 (72 hours from t0) hours from treatment (according to the experimental scheme shown in the scheme above). With the maximum dose of 10 μg/ml, 48 hours after t0, the cells are 50% fewer with respect to the untreated control. It has been observed that, if after the first 24-hour treatment, the OE is not re-administered, at 72 hours from to the M14 cells resume replication, while if the OE is re-administered at 48 hours from to, at 72 hours from to the cells are all detached from the plate and only cellular debris is observed by cytofluorimeter analysis.

Culture of Peripheral Blood Mononuclear Cells (PBMCs) from Healthy Donors and of MDMs and Treatment with C. zeylanicum EQ

The leukocyte-platelet layer, or buffy coat (BC), was obtained after centrifugation and separation of whole blood collected from healthy blood donors at the Immunohematology and Transfusion Medicine Unit, Policlinico Umberto I, University of Rome, Sapienza, Italy. BC was diluted 1:1 with phosphate buffered saline (PBS) and mononuclear cells separated on a Ficoll gradient (Eurobio, Paris, France). Cells were harvested, washed twice and seeded at a concentration of 2×10⁶/mL in culture flasks. Cultures were incubated in RPMI 1640 medium enriched with glutamine and supplemented with gentamicin (10 mg/L) and 20% foetal bovine serum (FCS), at 37° C. in an atmosphere of 5% CO₂-95% air. After 12 hours of adherence, the supernatant was discarded while the adherent macrophages were washed twice and peeled off with cold PBS by gentle scraping, then seeded at a concentration of 1×10⁶/mL.

MDM cells were treated on the seventh day of culture with three different dilutions of EO7 and of EO8, namely D1 (1:1000), D2 (1:10,000) and D3 (1:100,000, corresponding to 10 μg/mL), in RPMI medium. Cell viability was analysed after two hours by staining cells with Trypan blue. None of the dilutions tested revealed cytotoxic effects. The untreated MDMs were kept on a separate plate and in a different incubator to avoid any aerosol contamination of the volatile secondary metabolites present in the OE.

### Cell Cycle Analysis

A cell cycle analysis of M14 cells treated with C. zeylanicum EO7 was carried out by flow cytometry. In particular, after 24 hours of treatment with EO7, the cells were collected, washed in 1×PBS and fixed in 70% EtOH for at least one hour in a concentration of 1×10⁶ cells/mL. Untreated (CTRL) and treated cells were stained with a solution containing 50 μg/mL of Propidium Iodide (PI) and 75 KU/mL of RNase in 1×PBS for at least 30 minutes in the dark, to analyse the DNA content by flow cytometry. Approximately 20,000 events per sample were acquired using a FACSCalibur flow cytometer and CellQuest Pro software (Becton Dickinson). Percentages of cells at different cell cycle stages were estimated using linear DNA content histograms using the MODFIT software.

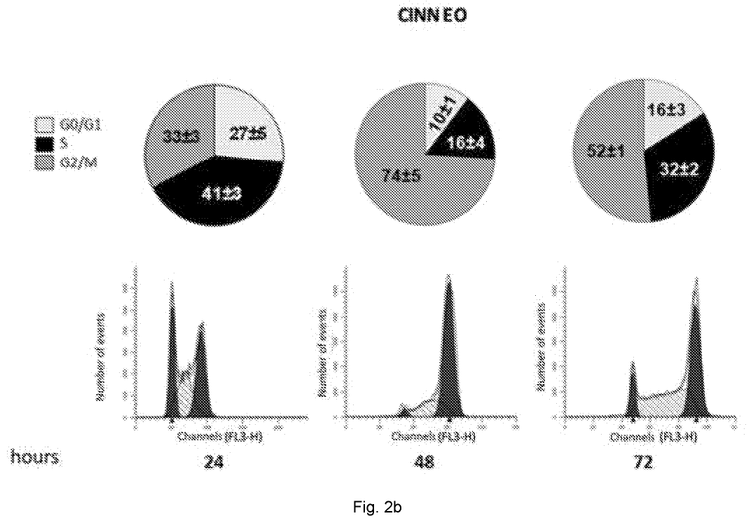

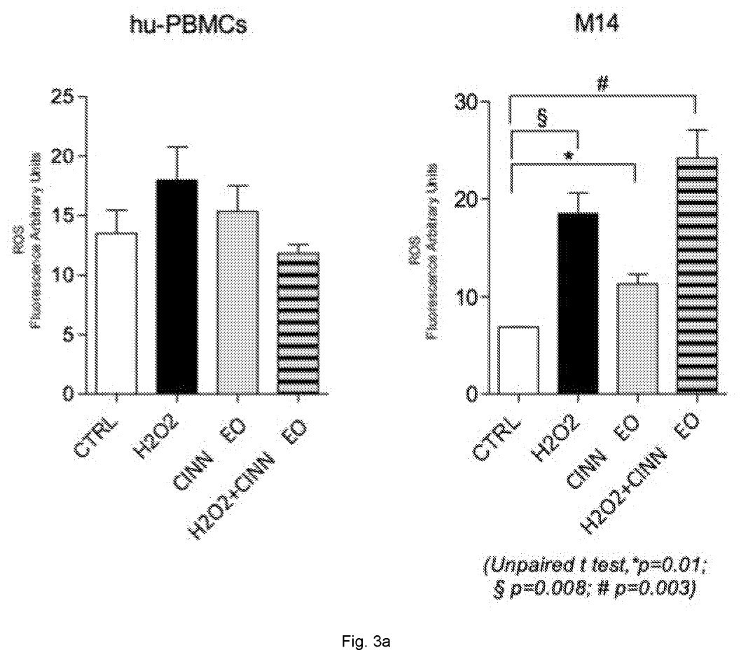

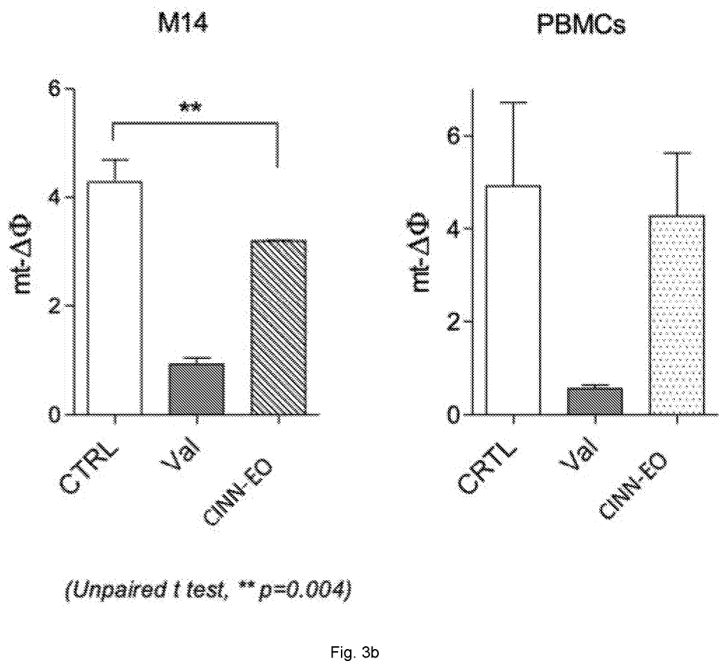

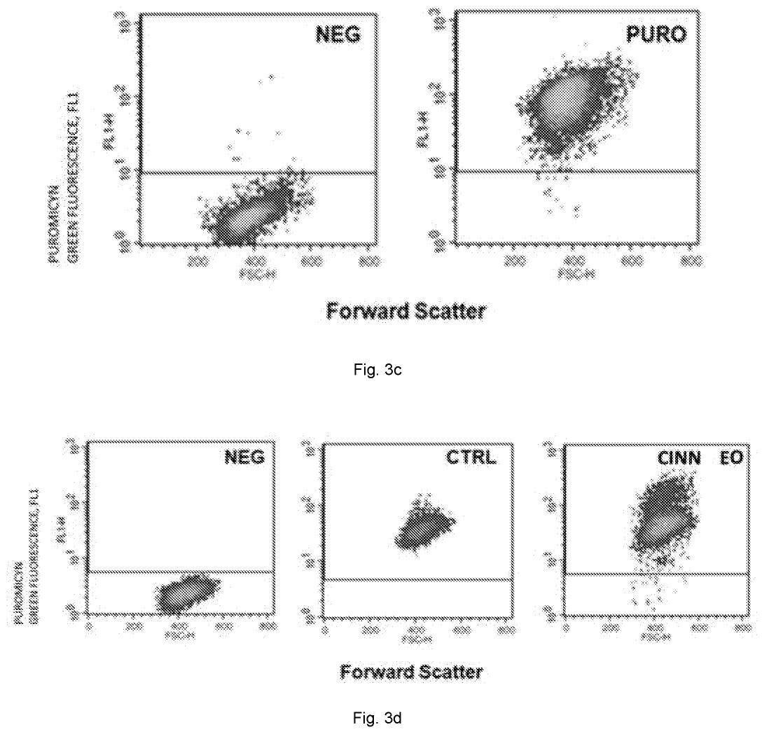

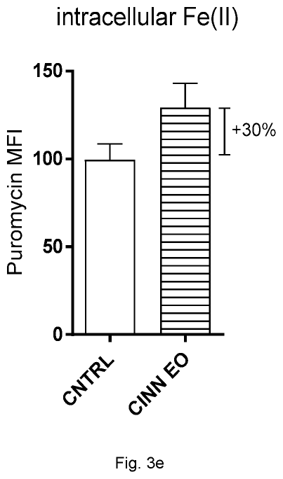

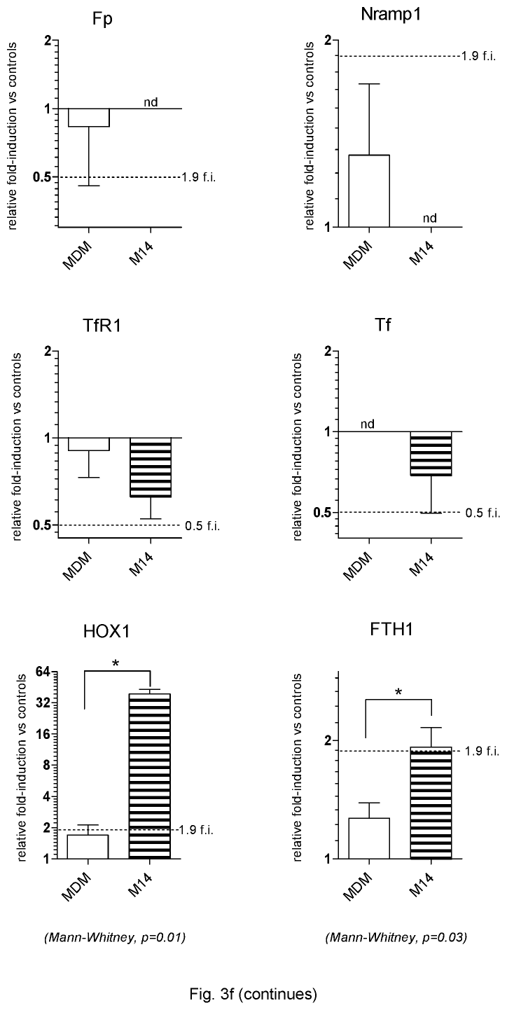

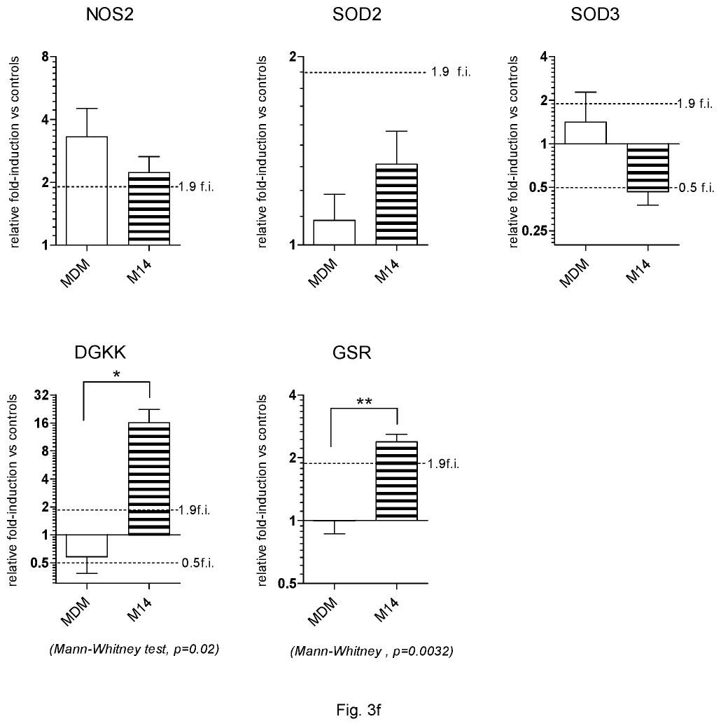

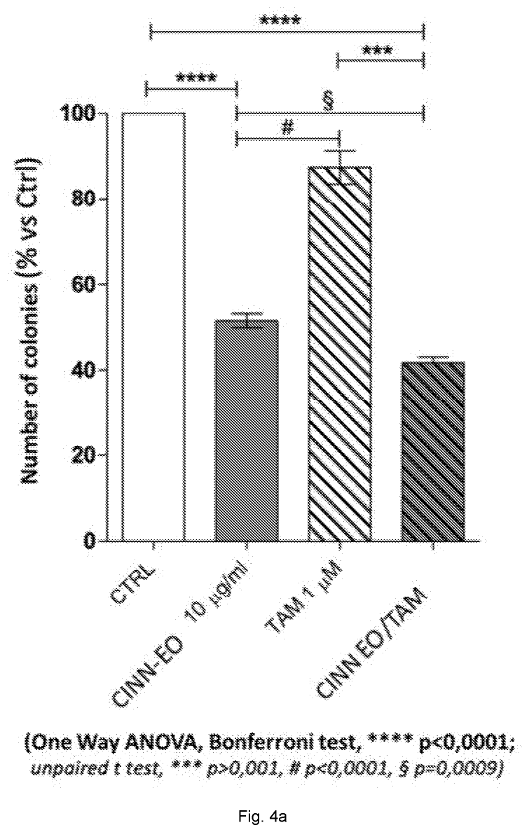

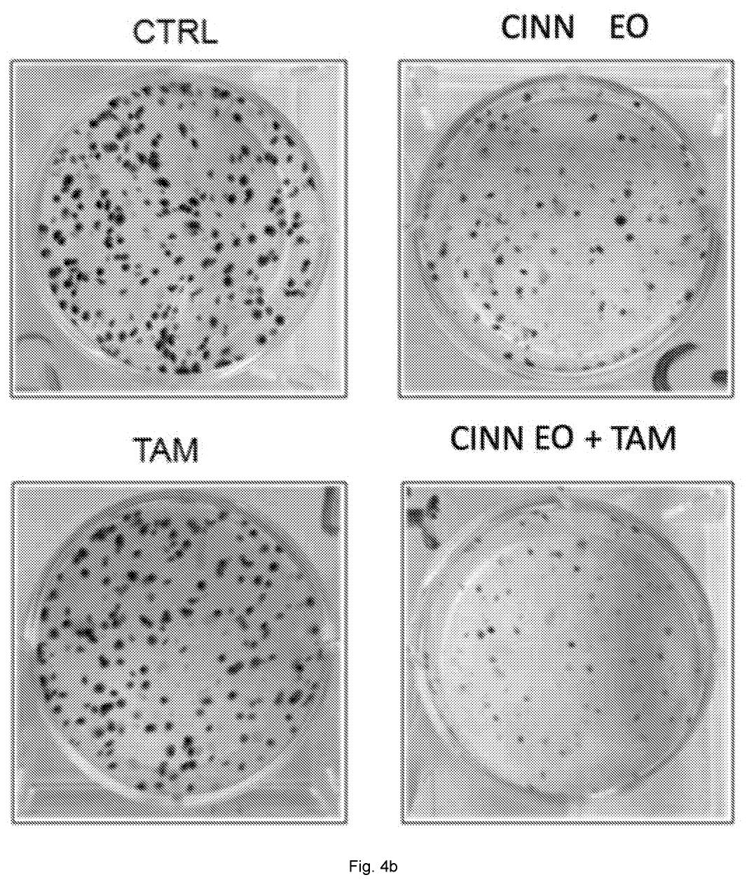

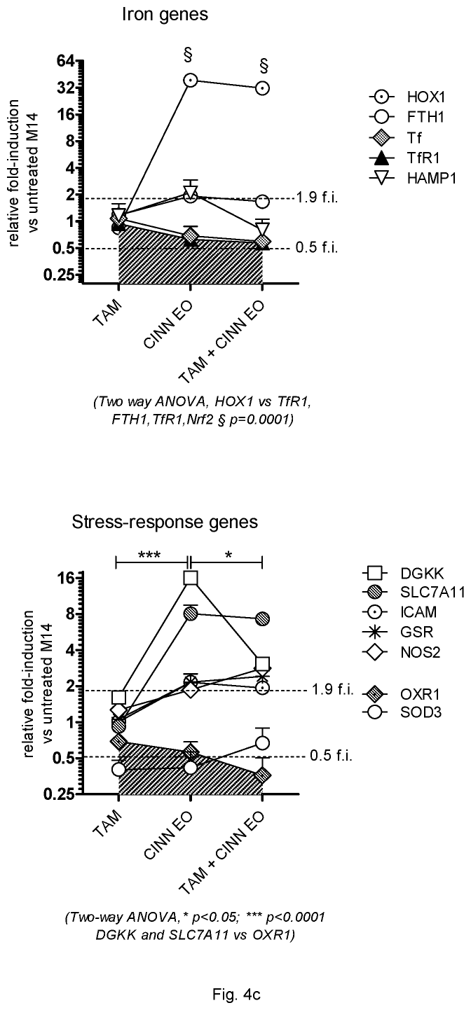

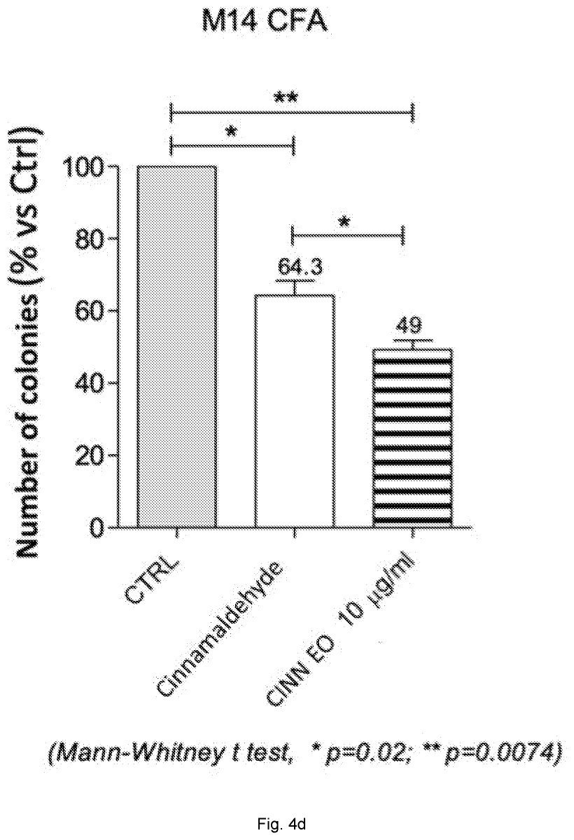

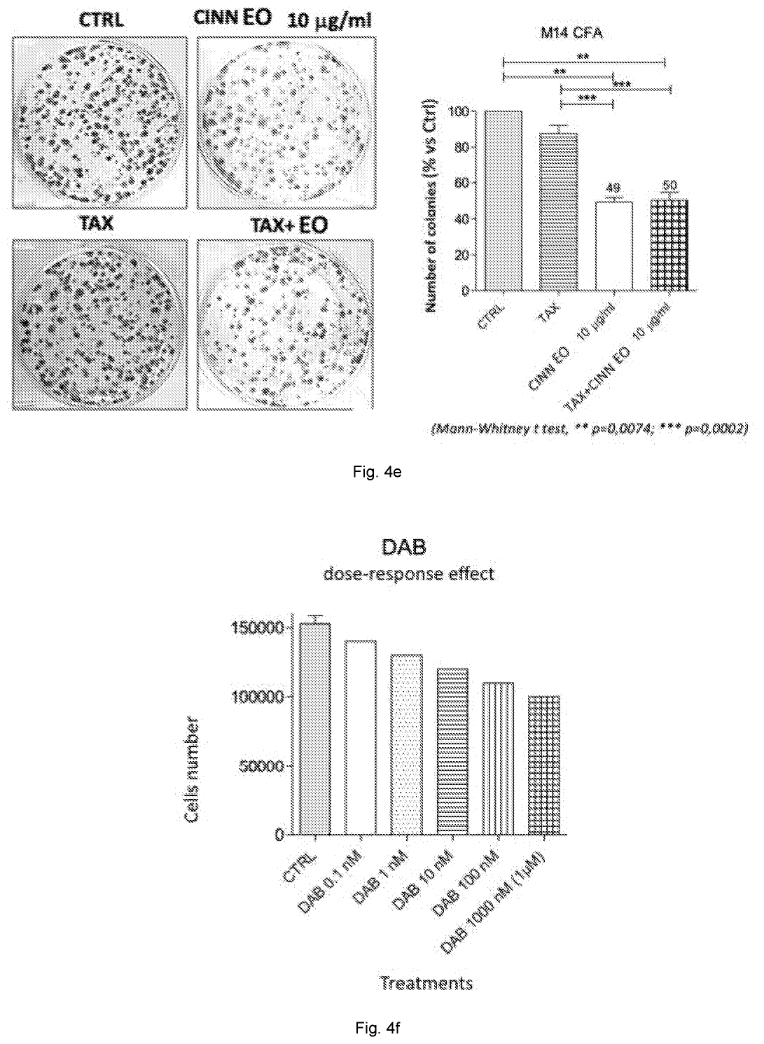

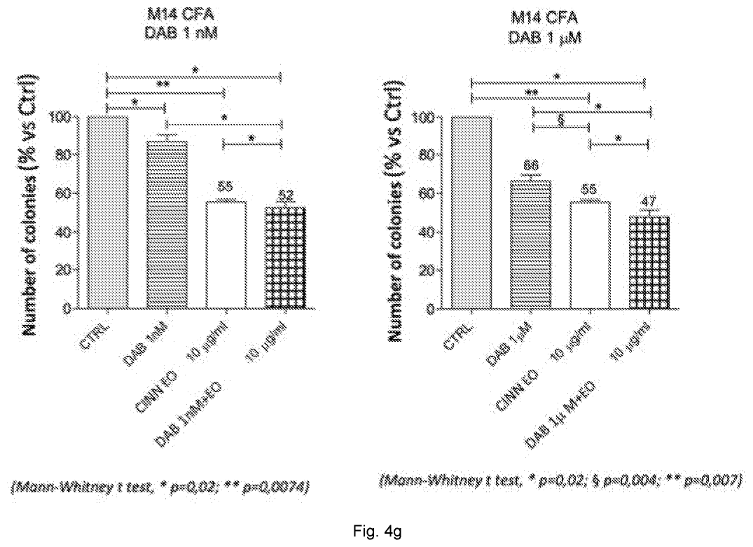

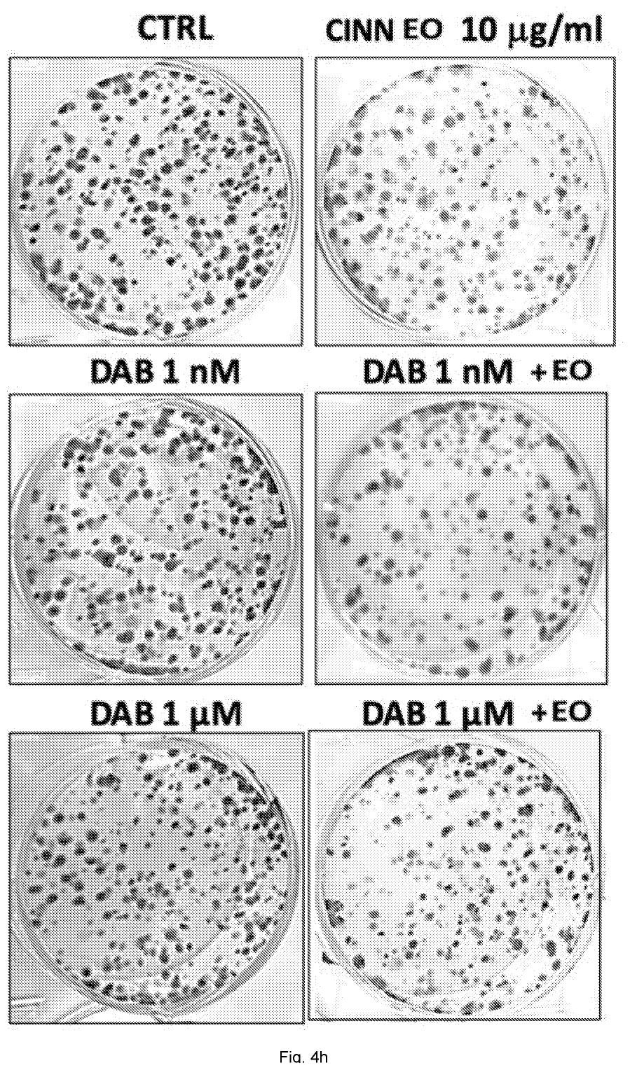

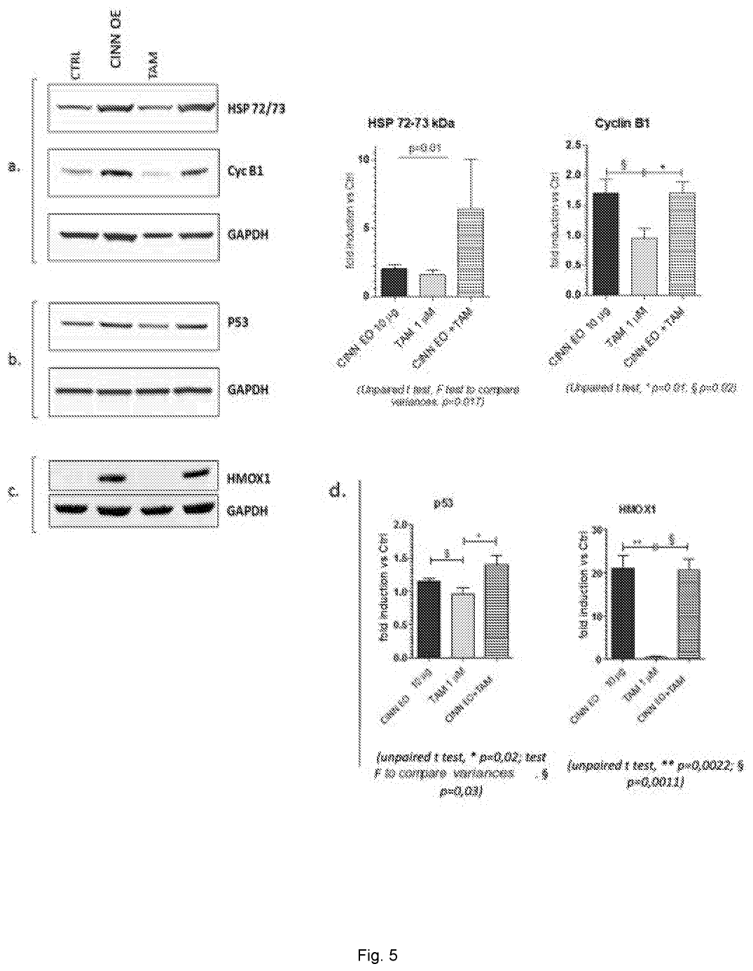

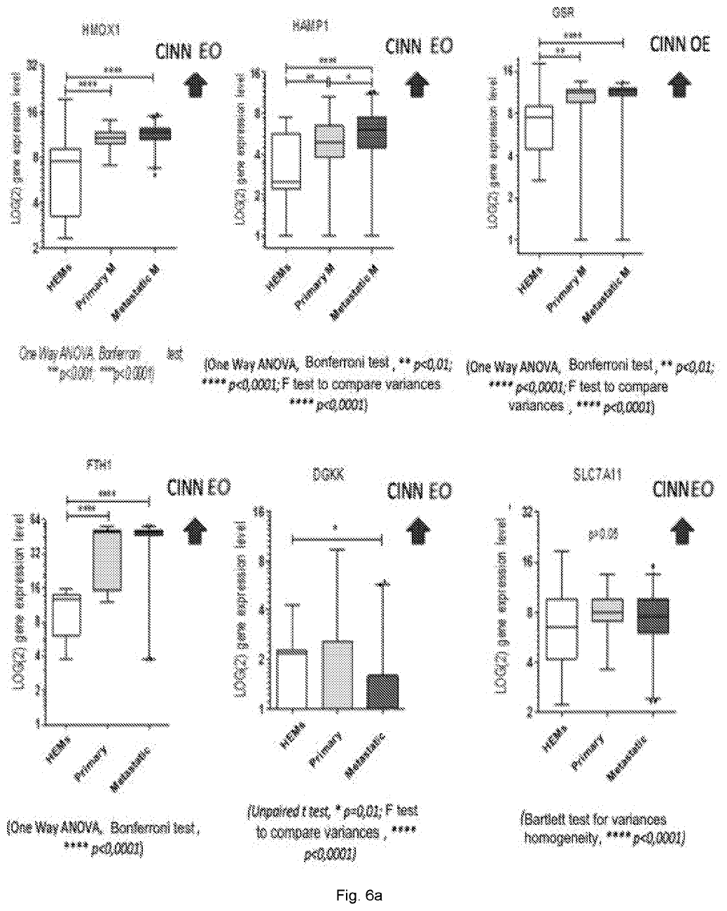

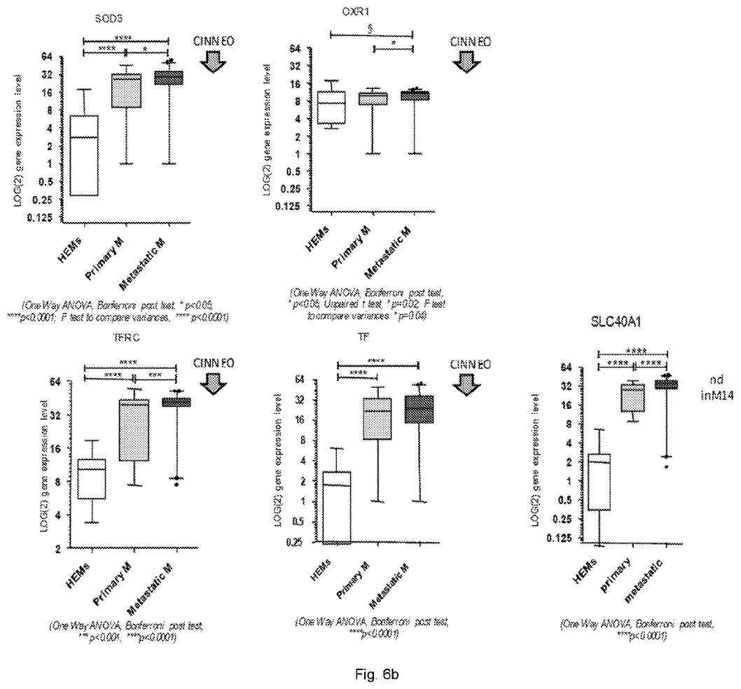

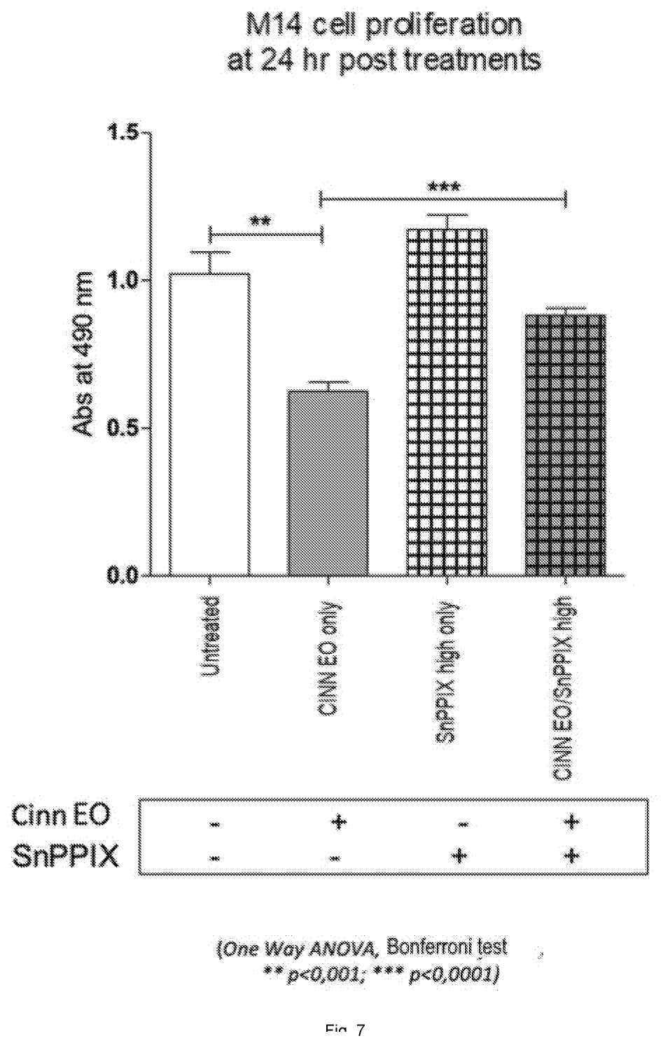

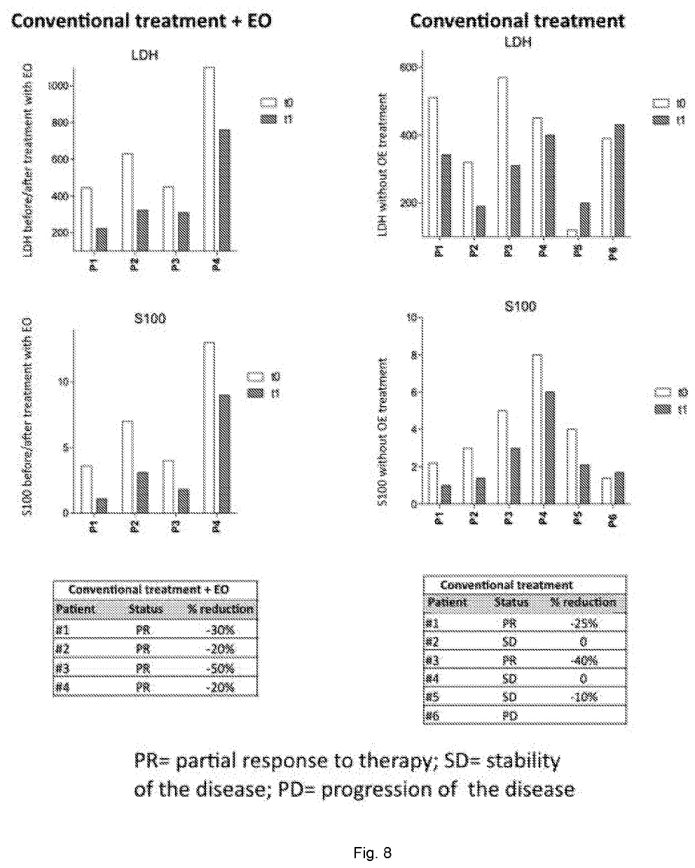

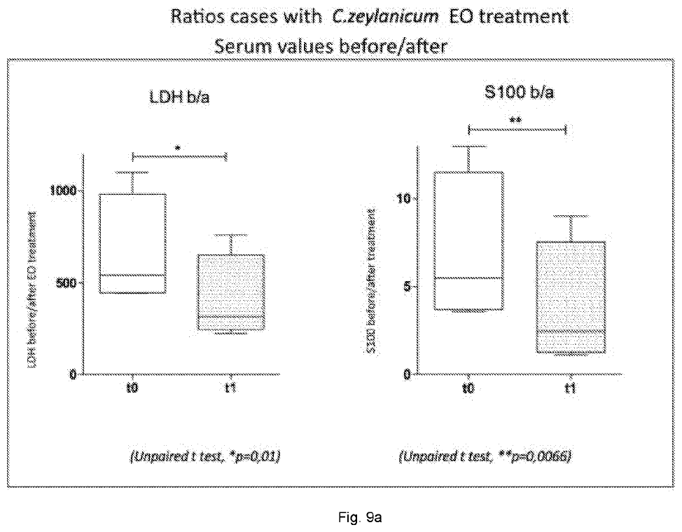

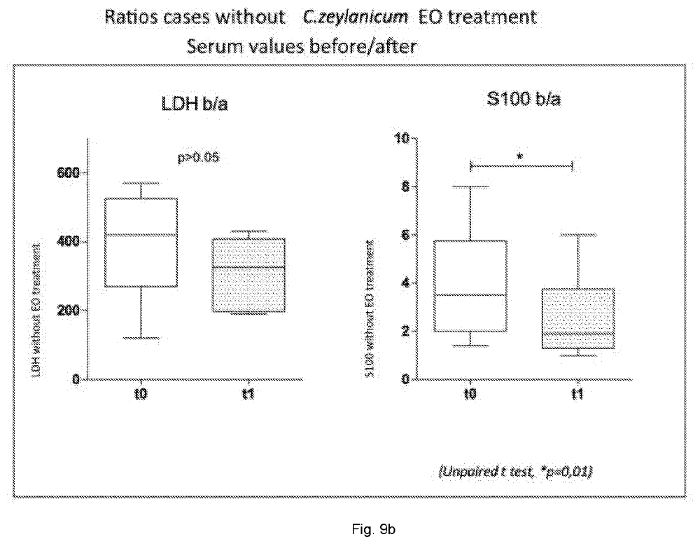

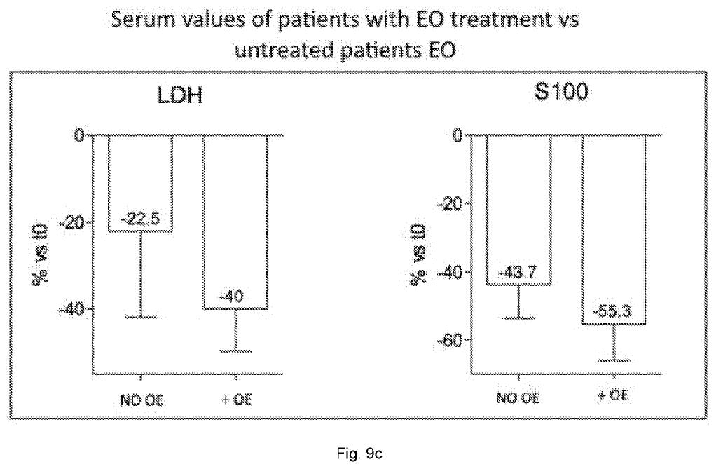

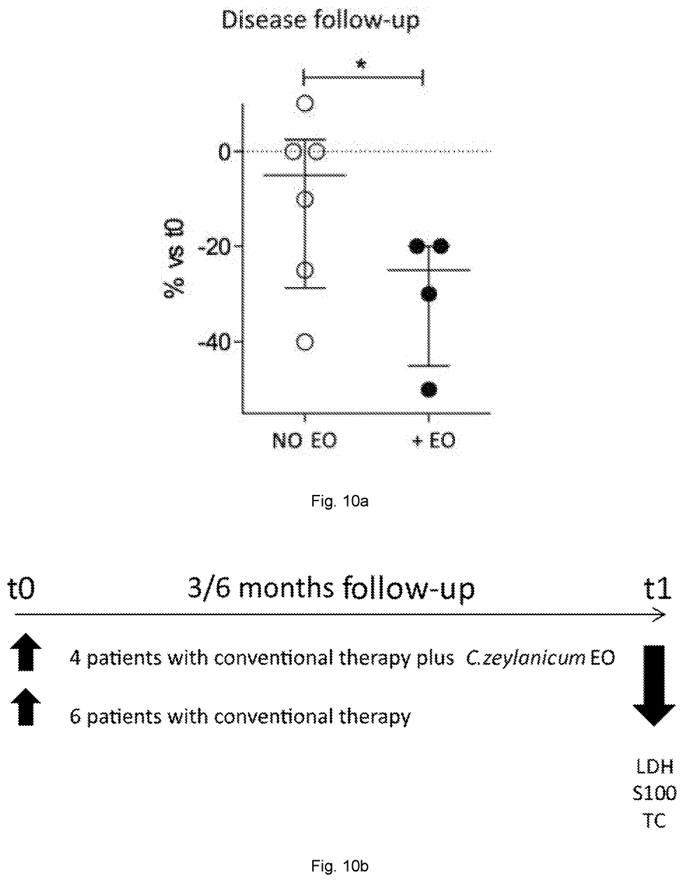

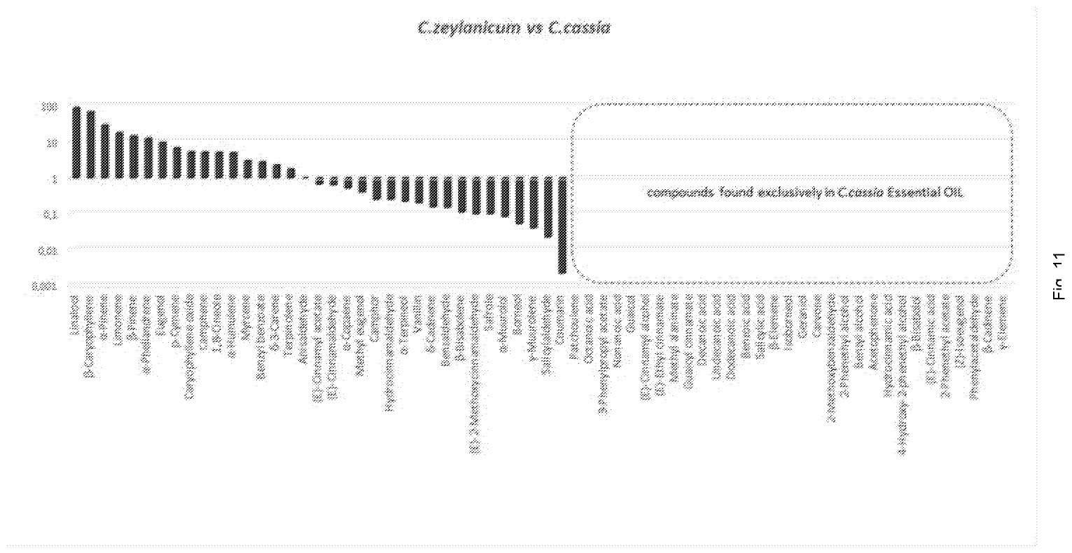

As shown in FIG. 2, the analysis of the cell cycle of the cells in culture revealed that 74% of the cells treated with C. zeylanicum EO7 are in the G2/M phase of the cell cycle, against 22% of the controls. The measurement of the DNA content of the treated cells therefore allows to assess that the treatment with C. zeylanicum EO induces the block of the cell cycle in G2/M, both 24 hours after treatment (CINN EO 74% vs CTRL 22%) and 48 hours after treatment (CINN EO 52% vs CTRL 21%). The checkpoint, or checkpoint, G2/M prevents the cell from entering mitosis. After 48 hours, this effect subsides, but by re-administering the OE to the M14 after the first 24 hours, the cells detach from the plate and die.

### ROS Analysis of the Intracellular Species

The generation of ROS species (reactive oxygen species) was analysed by FACS, using the probe sensitive to the oxidant 2 ‘, 7’-dichlorodihydrofluorescein (H2DCF-DA). The C. zeylanicum EO was administered at a dose of 10 μg/ml for 24 hours. The treated cells were then harvested and incubated with 10 μM of H2DCF-DA, in HBSS with 0.1% BSA, for 30 minutes, at 37° C. H₂O₂ at a concentration of 5 mM was used as a control for the generation of ROS. The cells were then washed in HBSS with 0.1% BSA and analysed by FACSCalibur flow cytometer and CellQuest Pro BD software. This assay was carried out in parallel with healthy donor whole blood mononuclear cells (PBMCs), which did not show any cytotoxicity following treatment with C. zeylanicum EO.

As shown in FIG. 3a, M14 cells treated with hydrogen peroxide (H₂O₂) and C. zeylanicum EO 7 and 8 show greater oxidative stress (intracellular amount of Reactive Oxygen Species, or ROS) than the mononuclear cells of the peripheral blood of healthy donors (Peripheral Blood Mononuclear Cells, or PBMCs).

### Analysis of the Mitochondrial Membrane Potential

The inventors carried out an analysis of the mitochondrial membrane potential Ad by FACS, using staining with JC-1 [Gatti et al. (2009) PlosOne 4 (5)]. After one washing in PBS, the cells were incubated with JC-1 at a concentration of 2.5 mg/mL for 20 minutes at room temperature, in the dark. After two washes in PBS, the samples were immediately analysed by FACSCalibur flow cytometer and CellQuest Pro (BD) software. As a control, a depolarized sample treated with the ionophore compound valinomycin for additional 15 minutes after staining with JC-1 was used. This assay was carried out in parallel with PBMCs from healthy donors.

As shown in FIG. 3b, M14 cells treated with C. zeylanicum EO show greater damage to the mitochondrial membrane than PBMCs from healthy donors. Valinomycin, used as a positive control of depolarization of the mitochondrial membrane, is an ionophore with high affinity for potassium, and is therefore able to modify the membrane potential of the mitochondrion, which undergoes swelling with consequent release of cytochrome C and damage to the mitochondrial respiratory chain.

Given the very high energy metabolism necessary for the replication of cancer cells, and the higher concentration of H₂O₂ in their cytoplasm, it is possible to assume that the pro-oxidant action of an electrophilic compound such as the C. zeylanicum, EO, by further increasing the concentration of intracellular ROS, has an effect on the pore permeability transition (PTP) of the mitochondrial membrane of these cells and affects its consequent depolarization

### Determination of the Intracellular Concentration of Ferrous Iron, or Iron2⁺, or Iron (II), or Metabolically Active (LIP) by Immunofluorescence Analysis of the Incorporation of Puromycin

An analysis of the content of metabolically active or labile iron (Labile Iron pool, or LIP), or ferrous iron, inside the OE-treated M14 cells, was carried out using a probe derived from trioxolane, puromycin, sensitive to Fe (II), as described by Renslo et al. Briefly, puromycin is conjugated with a 1,2,4-trioxolane scaffold to form an active cell probe Trx-Puro (3).

M14 cells were exposed to Puromycin (Sigma) or their conjugates (Trx-3-Puro, Trx-4-Puro) at the concentration suggested by Renslo et al. (1 μM, diluted in cell culture medium from a 1,000× stock solution in DMSO) for 4 hours. The cells were then harvested, washed with PBS and fixed in 4% PFA in 1×PBS for 10 minutes at room temperature. Then, after two washes with 1×PBS and a third wash with PBS containing 0.1% Triton X-100, the cells were incubated with the anti-Puromycin monoclonal antibody (PMY-2A4; Developmental Studies Hybridoma Bank) (1:500) in 10% FBS in PBS with 0.1% Triton X-100 for 30 minutes at 37° C. As a secondary antibody, an anti-mouse Alexa-Fluor 488 antibody was used. Cell-associated fluorescence was then analyzed by a FACSCalibur flow cytometer and CellQuest Pro (BD) software.

The obtained data, shown in FIG. 3c e 3d, show that C. zeylanicum EO-treated M14 cells have more intracellular ferrous iron (or Fe2+, or Fe II) than untreated controls. Ferrous or metabolically active iron, in the presence of H₂O₂, can give rise to a strong increase in oxygen radical species, through the Fenton reaction.

Gene Expression Analysis Using Quantitative Real Time PCR (qPCR)
RNA Extraction, cDNA Synthesis and qPCR Protocol

### RNA Extraction.

Adherent M14 cells were washed once with PBS and total RNA was extracted. Adherent cells were resuspended in 4M Guanidium iso-thiocyanate (GTC) lysing solution on ice. Total RNA was extracted as described in previous studies (Chomczynski & Sacchi, 2006), analysed for possible degradation on denaturing 1.5% agarose gel and quantified with UV spectroscopy at 260/280 nm.

Synthesis of cDNA (Reverse Transcription, RT) and qPCR.

One microgram of total RNA was retrotranscribed with random hexamers (pdN6) and SuperScript III Reverse Transcriptase (Invitrogen, Paisley, UK) according to the manufacturer's instructions. Quantitation of qPCR products was carried out with ABI PRISM 7500 FAST thermocycler (Thermo Fisher Scientific, USA). Real Master SYBR Green (Thermo Fisher Scientific, USA) was used to produce PCR products labelled with fluorochromes and we were able to measure the increase in fluorescent products during the repeated qPCR cycles. Primers for all amplicons (qPCR products) were designed with Primer-BLAST software (https://www.ncbi.nlm.nih.gov/tools/primer-blast). For all primers, the following cyclically repeated temperatures profile was: 2 min at 50° C. and 2 min at 95° C. followed by 1 min and 30 s at the specific temperature of each primer pair (for all primer pairs, the Annealing temperature chosen was 60° C., ±3° C.) for 40 cycles. L34 was used as an internal control because it was shown to remain stable under different experimental conditions. The relative expression level for each gene was calculated with the formula 2—ΔΔCt (Livak & Schmittgen, 2001).

An analysis of the expression of genes responsible for regulating iron metabolism and the intracellular stress response was conducted in both treated and control cells. As indicated in FIG. 3f, M14 cells treated with C. zeylanicum EO show a reduction in genes linked to transport (Transferrin, or Tf) and to the entry of Iron (Transferrin Receptor 1, or TfR1), which is an essential element for the replication and energy metabolism of cancer cells, while showing an increase in genes indicative of a stress response, such as Heme Oxygenase 1 (or HMOX1), Ferritin H (or FTH1), Glutathione S Reductase (or GSR) and Diacyl Glycerol Kinase Kappa (or DGKK). This suggests that the M14 treated with C. zeylanicum EO are in a state of intracellular oxidative stress, to which they try to respond with the induction of Superoxide Dismutase 2 and 3 (or SOD2 and SOD3) which, however, respectively, do not significantly increase, or do even decrease, compared to control cells. The treatment-induced stress response thus appears insufficient and incomplete.

Administration of C. zeylanicum EO in Combination with Tamoxifen (TAM), Taxol (TAX) and Dabrafenib (DAB).

M14 cells were treated with C. zeylanicum EO in 10 μg/ml concentration alone or in combination with TAM 0, 1 μM, ineffective dose, for 24 hours in order to evaluate the effect of C. zeylanicum OE in the treatment of M14 cells with the anticancer drug Tamoxifen. Adherent cells were hence harvested and seeded at clonal density (1,000 cells/plate) in 35 mm Petri dishes. Ten days after seeding, a solution of 2% methylene blue in 95% ethanol was added to the obtained monolayer and incubated for at least 30 minutes. The plates were then washed with ddH₂O and the colonies (at least 50 cells) were counted. The results were expressed as a percentage of colonies formed with respect to the number of cells seeded. Cell survival rate was calculated as % compared to control cells.

The data reported in FIGS. 4a and 4b show that the administration of C. zeylanicum EO in association with TAM, compared to the administration of TAM alone, sensitizes M14 cells to TAM, a drug to which M14s are normally resistant to the concentration used. M14 cells treated with TAM show no significant differences in survival compared to control, while cells treated with C. zeylanicum EO in combination with TAM are significantly less than the control, than the single treatment with TAM and than the same treatment with OE, although of little. The same data is obtained with the plate colony formation assay (or CFA), in which the number of live M14 cells after treatment with EO in association with TAM is lower than the control, of the single treatment with TAM and with C. zeylanicum EO. Also in this case, EO treatment and EO treatment in association with TAM show a reduction in the expression of transport (Tf) and iron entry (TfR1) genes, and an increase in genes indicative of a response to stress, such as HMOX1, FTH1, GSR and DGKK.

This suggests that the M14 treated with C. zeylanicum EO and TAM are in a state of intracellular stress, to which they could respond with the induction of SOD3 and of the oxidation resistance gene (or OXR1), which however, following treatment, decrease compared to control cells. The treatment-induced stress response seems, again, even in the presence of TAM, insufficient and incomplete.

Since cinnamaldehyde represents the most abundant bioactive compound in the EO object of the present invention, the inventors compared the cytotoxicity induced by cinnamaldehyde alone with that induced by OE (FIG. 4.d). Surprisingly, the experimental results shown in FIG. 4d showed a significant increase (p=0.02) of the cytotoxic effect of the OE object of the invention compared to that of cinnamaldehyde alone (survival 43.1% vs 64.3%, respectively).

A second experiment was conducted by administering C. zeylanicum OE in association with a second anticancer drug, Paclitaxel or Taxol (FIG. 4.e). The experiment showed the very poor induction of death in M14 cells by Taxol alone (less than 10%), while the administration of the combination increased cell death by more than 60% (p=0.0002). In this experiment, the difference between the cytotoxicity induced by EO alone and by the association was not statistically significant, although the latter showed a slight increase in cytotoxicity compared to OE alone.

Finally, we analyzed the administration of C. zeylanicum OE in association with the drug Dabrafenib (DAB).

In patients with melanoma in whom the presence in the genes of tumour cells of a specific mutation called “BRAF V600” has been ascertained, the drug Dabrafenib works by blocking BRAF, a protein kinase, involved in the stimulation of “protein kinases stimulated by mitogens” (ERK/MAPK: Mitogen Activated Protein Kinases), which participates in the stimulation of cell division. In melanomas with the BRAF V600 mutation, the mutated form of BRAF contributes to tumour development, allowing the uncontrolled division of cancer cells. By blocking the action of the mutated BRAF protein, Dabrafenib helps slow the growth and spread of the tumour. Dabrafenib is only given to patients with melanomas caused by the BRAF V600 mutation and approximately 50% of melanomas have mutations in the BRAF kinase, which is involved in the activation of MAPK.

To verify a possible potentiating effect of C. zeylanicum OE on DAB, we administered the OE in association with the drug to the M14 metastatic melanoma cells. FIG. 4.f. shows the dose-response effect of DAB on M14, and FIGS. 4.g and 4.h show the enhancing effect of C. zeylanicum OE on DAB used at 1 nM and 1 μM doses (significant differences between the combination and DAB alone, for both doses, p=0.02). tumorali di una specifica mutazione denominata “BRAF V600” il farmaco Dabrafenib agisce bloccando BRAF, una protein-chinasi, coinvolta nella stimolazione delle “proteinchinasi stimolate dai mitogeni” (ERK/MAPK: Mitogen Activated Protein Kinases), che partecipa alla stimolazione della divisione cellulare. Nei melanomi con mutazione BRAF V600 la forma mutata di BRAF contribuisce allo sviluppo del tumore, permettendo la divisione incontrollata delle cellule tumorali. Bloccando l'azione della proteina BRAF mutata, il Dabrafenib aiuta a rallentare la crescita e la diffusione del tumore. Il Dabrafenib è somministrato solo ai pazienti con melanomi causati dalla mutazione BRAF V600 e circa il 50% dei melanomi presenta mutazioni della chinasi BRAF, coinvolta nella attivazione di MAPK.

Per verificare un possibile effetto potenziante dell'OE di C. zeylanicum sul DAB, abbiamo somministrato l'OE in associazione con il farmaco alle cellule di melanoma metastatico M14. La FIG. 4.f. mostra l'effetto dose-risposta del DAB sulle M14, e le FIGS. 4.g e 4.h mostrano l'effetto potenziante dell'OE di C. zeylanicum sul DAB usato alle dosi 1 nM e 1 μM (differenze significative tra la associazione e il DAB da solo, per entrambe le dosi, p=0.02).

### Western Blot Analysis

Cultured cells were washed twice with 1×PBS and then incubated for 1 minute in urea-based buffer (8 M urea, 100 mM NaH₂PO₄ and 10 mM Tris pH 8), harvested and briefly sonicated (10 sec). The proteins were subjected to SDS-polyacrylamide gel electrophoresis. The resolved proteins were transferred by blotting carried out overnight on nitrocellulose membranes, which were then treated in 1×PBS containing 5% fat-free milk, for at least 1 hour. The blots were incubated with the following primary anti-human antibodies: rabbit polyclonal anti-cyclin B1 (H433; Santa Cruz); anti-P53 mouse monoclonal antibody (DO-7; Dako); mouse monoclonal anti-transferrin antibody (clone #507506; research and development systems); mouse monoclonal antibody anti-HMOX1 (sc-136960; Santa Cruz Biotechnology); mouse monoclonal anti-GAPDH antibody (6C5; Millipore); and anti-HSP 72/73 mouse monoclonal antibody (Ab1-W27; Oncogene Science Inc.). The membranes were then incubated for 45 minutes with the appropriate secondary antibody: donkey anti-rabbit secondary antibody IRdye800 (LI-COR) or donkey anti-mouse secondary antibody IRdye800 (LI-COR). The membranes were then analysed with a Licor Odyssey Infrared Image System in the infrared channel at 800 nm.

In Silico Analysis of Gene Expression Profile in Normal Melanocytes, Primary and Metastatic Tumor Tissue from Patients Included in Public Databases, Such as the Cancer Genome Atlas (TCGA) and Gene Expression Omnibus (GEO).

An analysis of the C. zeylanicum EO-induced gene expression pattern was carried out, this was then compared with the expression patterns of the same genes in healthy donor human melanocytes (HEMs) and in patients with primary melanoma (HPM) and metastatic (HMM), deposited in gene expression databases (homogeneous data generated on the Affymetrix platform were chosen) such as Gene Expression Omnibus Database (https://www.ncbi.nlm.nih.gov/geo/), in the absence of drug treatments and/or specific mutations. As evident from FIG. 6, this comparison showed a significant divergence between the expression pattern induced by EO treatment and the progression from Melanocyte to Primary Melanoma to Metastatic Melanoma, in particular for some stress response genes. The group of genes repressed by the C. zeylanicum EO treatment on M14 cells appears instead to be increased in the progression to Primary and Metastatic Melanoma.

This finding, associated with disease progression, is in line with the generally pro-oxidized state of cancer cells, which therefore need anti-oxidant genes in order not to be damaged by an excessive load of oxidative stress, and by the need for ferrous iron for their very high energy metabolism.

In this context, the decrease in genes induced by the C. zeylanicum EO treatment would prevent both the reduction of intracellular stress and the very delicate balance in the intracellular concentration of ferrous iron, and could therefore interfere in this way with cell replication and the consequent block in G2/M.

Inhibitory Effect of SnPPIX HMOX1 on M14 Cell Proliferation after Treatment with OE.

The HMOX1 protein plays a central role in cell death by ferroptosis and in the regulation of intracellular Fe (II). To verify the role of the HMOX1 protein in the death of metastatic M14 melanoma cells following treatment with OE, the metabolic activity and proliferation of M14 cells were determined, in the presence and absence of tin protoporphyrin IX (SnPPIX), an enzyme that inhibits HMOX-1. The analysis was carried out by CellTiter 96® (Promega) cell proliferation colorimetric assay, after administration of OE. Melanoma cells were incubated with 10 μg/mL EO alone or in combination with 5 UM SnPPIX for 24 hours. As a control, the cells were also incubated with 5 μM SnPPIX alone. After treatment, cells were incubated with MTS, [3-(4,5-dimethylthiazol-2-yl)-5-(3-carboxymethoxyphenyl)-2-(4-sulfophenyl)-2H-tetrazolium, and with an electron coupling reagent (phenazine methosulfate), at 37° C. for approximately 1 hour, following the manufacturer's instructions. Metabolically active cells reduce MTS to a soluble formazan product, the absorbance of which is measured at 490 nm in growth medium. The absorbance values of the collected samples were subtracted from the background absorbance of the control medium.

As indicated by the data in FIG. 7, the experiment shows that inhibition of the HMOX1 protein cancels the cytotoxic effect of OE on M14 cells. This data confirms the hypothesis of the possible cell death of M14 by Ferroptosis. M14 treated with C. zeylanicum EO and HMOX-1 inhibitor, in fact, show a total recovery of metabolic activity, compared to those treated with C. zeylanicum EO alone.

Case Reports of Ten Patients with Metastatic Melanoma Treated and not Treated with C. zeylanicum EO in Addition to Conventional Therapies.

A group of patients with metastatic melanoma (n=4), signed the informed consent, decided to participate in case studies (positive arm) in which six drops of C. zeylanicum EO were added to conventional therapy (composition 07 of the Table 1), divided into three doses of two drops to be taken after main meals (breakfast, lunch and dinner) diluted in a glass of yogurt or fruit juice (given the possible sensitizing effect of EO on the gastric mucosa, already challenged by chemotherapeutic agents). The follow-up period lasted for all from 3 to 6 months (time 1, t1) starting from the beginning of treatment. A control group of patients with the same disease, enrolled prior to the current trial (negative arm), received only conventional therapy, with the same follow-up period (see FIG. 10.b).

Table 2 below shows all the general data relating to the two groups of patients.

| | | | | braf | Pharmacological therapy | | | | |
|---|---|---|---|---|---|---|---|---|---|
| | | | | v600e | First | Second | Third | LDH | LDH |
| Melanoma | Metastasis | Gender | Age | mutation | line | line | line | t0 | t1 |

Positive arm (conventional therapy plus EO of C. zeylanicum)

| | | | | | | | | | |
|---|---|---|---|---|---|---|---|---|---|
| Patient 1 | Liver | female | 52 | present | Dabrafenib + | | | 445 | 223 |
| | lymphnodes | | | | Trametinib | | | | |
| Patient 2 | brain | male | 63 | present | Dabrafenib + | Nivolumab | | 630 | 322 |
| | bones | | | | Trametinib | | | | |
| Patient 3 | liver | female | 42 | absent | Nivolumab | | | 450 | 310 |
| | lymphnodes | | | | | | | | |
| Patient 4 | lungs | male | 69 | present | Dabrafenib + | Nivolumab, | Temazolomide | 1100 | 760 |
| | | | | | Trametinib | no | | | |

Negative arm (conventional therapy)

| | | | | | | | | |
|---|---|---|---|---|---|---|---|---|
| Patient 1 | liver | female | 43 | present | Dabrafenib + | | 510 | 342 |
| | | | | | Trametinib | | | |
| Patient 2 | brain | male | 71 | absent | Pembrolizumab | | 320 | 190 |
| Patient 3 | LN and | male | 61 | present | Dabrafenib + | | 570 | 310 |
| | bones | | | | Trametinib | | | |
| Patient 4 | liver and | male | 57 | absent | Nivolumab | | 450 | 400 |
| | spleen | | | | | | | |
| Patient 5 | cutaneous | female | 60 | absent | Pembrolizumab | | 120 | 200 |
| | diffused | | | | | | | |
| Patient 6 | | female | 53 | absent | Nivolumab | Temazolomide | 390 | 430 |

| | LDH | % | | | S100 | % | |
|---|---|---|---|---|---|---|---|
| | t1 vs t0 | LDH | S100 | S100 | t1 vs t0 | S100 | Disease |
| Melanoma | (%) | reduction | t0 | t1 | (%) | reduction | reduction |
| Positive arm (conventional therapy plus EO of C. zeylanicum) | | | | | | | |
| Patient 1 | 50.11 | 49.89 | 3.6 | 1.1 | 30.56 | 69.44 | 30% |
| Patient 2 | 51.11 | 48.89 | 7 | 3.1 | 44.29 | 55.71 | 20% |
| Patient 3 | 68.89 | 31.11 | 4 | 1.8 | 45.00 | 55.00 | 50% |
| Patient 4 | 69.09 | 30.91 | 13 | 9 | 69.23 | 30.77 | 20% |
| Negative arm (conventional therapy) | | | | | | | |
| Patient 1 | 67.06 | 32.94 | 2.2 | 1 | 45.45 | 54.55 | 25% |
| Patient 2 | 59.38 | 40.63 | 3 | 1.4 | 46.67 | 53.33 | TC at 3 months |
| | | | | | | | radiological |
| | | | | | | | stability of the |
| | | | | | | | lesions |
| Patient 3 | 54.39 | 45.61 | 5 | 3 | 60.00 | 40.00 | TC at 3 months |
| | | | | | | | 40% reduction |
| | | | | | | | of the lesions |
| Patient 4 | 88.89 | 11.11 | 8 | 6 | 75.00 | 25.00 | At 4 months |
| | | | | | | | stabilisation of |
| | | | | | | | the disease |
| Patient 5 | 166.87 | −66.67 | 4 | 2.1 | 52.5 | 47.5 | After 4 months |
| | | | | | | | 10% disease |
| | | | | | | | reduction |
| Patient 6 | 110.26 | −10.26 | 1.4 | 1.7 | 121.43 | −21.43 | After 4 months |
| | | | | | | | progression, |
| | | | | | | | after second |
| | | | | | | | line |
| | | | | | | | progression |

Table 2: General data on the patient population included in the study.

Table 3 shows the results of the response to the two treatments in terms of patient status and percentage reduction of lesions at three/six months following CT. These data show a greater reduction in lesions in patients also treated with OE.

TABLE 3
Response to treatment (3/6 months follow up)

| Conventional treatment plus OE | | | Conventional treatment | | |
|---|---|---|---|---|---|
| Patient | Status | % reduction | Patient | Status | % reduction |
| #1 | PR | −30% | #1 | PR | −25% |
| #2 | PR | −20% | #2 | SD | 0 |
| #3 | PR | −50% | #3 | PR | −40% |
| #4 | PR | −20% | #4 | SD | 0 |
| | | | #5 | SD | −10% |
| | | | #6 | PD | |

Key to patient response to treatment, based on lesion reduction (%): partial response (PR) when ≥20%, Stable disease (SD) when ≤20%, Progressive disease (PD).

Table 3: Treatment response results of patients with metastatic melanoma. A first group of patients (n=4) received conventional therapies plus six drops/day of C. zeylanicum EO, at three different time periods (after meals at breakfast, lunch and dinner); a second group of patients (n=6), previously enrolled, received only conventional therapies.

The progress of the therapy was monitored with the measurement of two serum biomarkers used in clinical practice, lactate dehydrogenase (LDH) and protein S100. Serum lactate dehydrogenase (LDH) has, since 2002, been included as a serological prognostic marker in stage IV metastatic melanoma according to the classification of the American Joint Committee on Cancer (AJCC, DOI: 10.1111/j. 1468-3083.2011.04210.x).

S100 is a calcium-binding protein with a molecular weight of 21,000 found in the nervous system of vertebrates (particularly in astrocytes). In previous studies, the S100 protein was found in malignant melanoma tissue, where it was completely absent from normal skin samples and normal lymph nodes. Its use for the diagnosis of metastatic melanoma is acknowledged by the scientific community (DOI: 10.1111/j. 1468-3083.2011.04210.x) FIG. 8 shows the LDH and S100 trend, in response to therapeutic treatment in the follow-up period (3/6 months) and the association with the relative % reduction in lesions at t1. The left panels show the results obtained in patients also treated with C. zeylanicum OE, while the right ones show the patients treated with conventional therapy alone. It is clear how the addition of EO to conventional therapy significantly reduces the values of the two prognostic markers in all the examined patients.

FIG. 9 shows the results of the statistical analysis of the differences in LDH and S100 values in the two patients groups. Box a. describes the trend of the two biomarkers in patients treated with conventional therapy plus OE. The differences between t0 and t1 (3/6 months of follow up) were significant (LDH p=0.01, S100 p=0.0066). Box b. describes the trend of the two biomarkers in patients treated with conventional therapy only. The differences between t0 and t1 (3/6 months of follow up) were significant only for S100, with a much lower p value (p=0.01 vs p=0.0066). Box c. compares the variations in t1vs t0 between the two groups. The differences between these ratios, although not statistically significant, show a clear trend towards a greater reduction in LDH over the course of 3/6 months of treatment with EO added to conventional therapies.

Finally, FIG. 10 shows the results of the statistical analysis of the differences in the % reduction of metastatic lesions between the two patients' groups (as described in Table 2 and Table 3). Although these are a limited number of cases, the therapeutic effect of administering C. zeylanicum OE to conventional therapies seems evident to us. This EO was not administered at the maximum doses allowed by the EMA (see point 10 page 14, 10 May 2011 EMA/HMPC/706229/2009. Committee on Herbal Medicinal Products (HMPC) Community herbal monograph on Cinnamomum verum J.S. Presl, corticis aetheroleum), given the possible sensitization of the gastric mucous membranes of patients by the same OE. The delivery of this EO in gastro-resistant capsules will make it possible to use the maximum dose of 10 drops/day allowed by the EU regulatory authority, with a reasonable expected improvement in the therapeutic effects on patients.

Comparison Between Composition of C. zeylanicum E08 as Disclosed in Table 1 and of C cassia According to Li et al 1998.

Table 11.7 of Li et al 1998 reported below as table 4, provides the results of the analysis of leaf oil and bark oil of C. cassia.

TABLE 4

| Compound | Leaf oil (%) | Bark oil (%) |
|---|---|---|
| α-Pinene | 0.05-0.36 | 0.10-0.25 |
| Camphene | 0.04-0.05 | 0.05-0.10 |
| β-Pinene | 0.04-0.15 | 0.14-0.22 |
| Myrcene | 0.02-0.03 | t-0.10 |
| α-Phellandrene | 0.01-0.03 | t-0.13 |
| Limonene | 0.13-0.24 | 0.14-0.29 |
| 1,8-Cineole | 0.05-0.08 | 0.06-1.07 |
| δ-3-Carene | 0.03-0.05 | t-0.07 |
| p-Cymene | 0.11-0.19 | 0.04-0.18 |
| Camphor | 0.07-0.15 | 0-0.08 |
| Benzaldehyde | 1.42-1.48 | 0.50-1.10 |
| Linalool | 0.11-0.23 | 0.08-0.16 |
| Terpinolene | t | 0-0.04 |
| β-Caryophyllene | 0.16-0.20 | t-0.27 |
| α-Humulene | t-0.03 | 0-0.15 |
| β-Elemene | — | t-0.06 |
| Isoborneol | 0-0.20 | 0-0.27 |
| Borneol | 0.15-0.41 | 0.06-1.27 |
| α-Terpineol | t-0.10 | 0.07-2.05 |
| Geraniol | t | 0.08-0.31 |
| Carvone | 0.57-0.64 | 0-0.34 |
| 2-Methoxybenzaldehyde | 0.08 | 0-0.12 |
| Safrole | — | t-0.20 |
| γ-Elemene | 0-t | 0-0.41 |
| δ-Cadinene | t | t-0.13 |
| β-Cadinene | — | t-0.10 |
| Hydrocinnamaldehyde | 0.88-0.89 | 0-0.24 |
| Phenylacetaldehyde | 0.07-0.16 | t-0.27 |
| Methyl eugenol | 0.14-0.15 | t-0.05 |
| (E)-Cinnamaldehyde | 64.10-68.30 | 80.40-88.50 |
| α-Copaene | 0.41-0.49 | 0.23-0.68 |
| Vanillin | t | t-0.10 |
| Salicylaldehyde | 0.05-0.42 | 0.04-0.85 |
| 2-Phenethyl alcohol | 0.11-0.27 | t-0.16 |
| Benzyl alcohol | t-0.05 | — |
| Acetophenone | t-0.1 | 0-0.6 |
| Eugenol | 0.04-0.06 | 0.03-1.08 |
| (Z)-Isoeugenol | 0.14-0.28 | 0.12-0.66 |
| (E)-Cinnamyl acetate | 4.50-12.50 | 0.60-5.10 |
| γ-Muurolene | t | t-0.50 |
| Anisaldehyde | 0.58-1.02 | t |
| 2-Phenethyl acetate | t-1.55 | — |
| β-Bisabolene | t-0.06 | t-0.18 |
| β-Bisabolol | t | t-0.35 |
| α-Muurolol | 0-0.08 | 0-0.24 |
| Coumarin | 0.03-0.08 | t-0.45 |
| (E)-Cinnamic acid | 0.80-2.48 | 0.12-3.10 |
| (E)-2-Methoxycinnamaldehyde | 8.40-10.50 | t-2.50 |
| Hydrocinnamic acid | 0.18-0.51 | 0.024 |
| 4-Hydroxy-2-phenethyl alcohol | 0-0.12 | 0-0.10 |
| Caryophyllene oxide | 0.15-0.17 | 0-0.10 |
| Patchoulene | 0.06-0.07 | 0-0.04 |
| Octanoic acid | t | 0-t |
| 3-Phenylpropyl acetate | 0.21-0.43 | 0.05-0.22 |
| Nonanoic acid | t-0.10 | 0-t |
| Guaicol | t | 0-0.08 |
| (E)-Cinnamyl alcohol | 0.15 | 0.05-0.13 |
| (E)-Ethyl cinnamate | 0.11-0.27 | t-0.14 |
| Benzyl benzoate | 0.07-0.15 | t-0.38 |
| Methyl alaninate | t-0.05 | — |
| Guaicyl cinnamate | t | t |
| Decanoic acid | t | 0-t |
| Undecanoic acid | 0-0.05 | 0-0.11 |
| Dodecanoic acid | t-0.04 | 0-t |
| Benzoic acid | 0.07-0.11 | 0.07-0.10 |
| Salicylic acid | t-0.10 | 0.10-0.20 |

Bark EO composition of both species was compared, and the ratio between the percentage of each compound in C. zeylanicum EO8 of the invention and the same compound in C. cassia according to Li et al 1998 was calculated and is reported in FIG. 11.

The figure shows a strong difference between the two oils, in particular an extremely relevant difference in coumarin and safrole content.

1. An essential oil of Cinnamomum zeylanicum, wherein said essential oil comprises:
E-cinnamaldehyde from 50 to 58%, and
eugenol from 3 to 8%.

2. The essential oil of Cinnamomum zeylanicum according to claim 1, wherein said essential oil comprises
monoterpene hydrocarbons from 12 to 14.5%,
oxygenated monoterpenes from 13 to 14.5%,
sesquiterpenes from 9 to 11%,
E-cinnamaldehyde from 50 to 58%,
eugenol from 3 to 8%.

3. The essential of Cinnamomum zeylanicum according to claim 1, wherein said oxygenated monoterpenes are from 13.4 to 14.3%.

4. The essential of Cinnamomum zeylanicum according to claim 1, wherein said sesquiterpenes are from 9.7 to 10.5%.

5. The essential of Cinnamomum zeylanicum according to claim 1, wherein said E-cinnamaldehyde is from 50 to 57%.

6. The essential of Cinnamomum zeylanicum according to claim 1, wherein said eugenol is from 3.5 to 7.5%.

7. The essential of Cinnamomum zeylanicum according to claim 1, further comprising
o-methoxy cinnamaldehyde from 0.08 to 0.2% and/or
E-Cinnamyl acetate from 1.2 to 2.5%.

8. The essential of Cinnamomum zeylanicum according to claim 1, wherein said essential oil comprises:
monoterpene hydrocarbons from 12 to 14.5%,
oxygenated monoterpenes from 13.44 to 14.30%,
sesquiterpenes from 9.73 to 10.46%,
E-cinnamaldehyde from 50.55 to 56.55%,
eugenol from 3.74 to 7.03%,
o-methoxy cinnamaldehyde from 0.09 to 0.16%,
E-Cinnamyl acetate from 1.37 to 2.41%.

9. The essential of Cinnamomum zeylanicum according to claim 1, wherein said essential oil comprises:
14.02% of monoterpene hydrocarbons,
13.44% of oxygenated monoterpenes,
9.73% of sesquiterpenes,
56.55% of E-cinnamaldehyde,
3.74% of eugenol,
0.16% of o-methoxy cinnamaldehyde; and
1.37% of E-Cinnamyl acetate.

10. The essential of Cinnamomum zeylanicum according to claim 1, wherein said essential oil is extracted by vapor stream.

11. The essential oil of Cinnamomum zeylanicum according to claim 1, wherein said oil does not comprise coumarin, or comprises coumarin<0.0005%.

12. The essential oil of Cinnamomum zeylanicum according to claim 1, wherein said oil comprises safrole<0.1%, preferably <0.05%.

13. The essential of Cinnamomum zeylanicum according to claim 1, wherein said essential oil is extracted from the bark of young branches of Cinnamomum zeylanicum plants older than 2 years.

14. A method of treating melanoma in a patient in need thereof, comprising administering to the patient a therapeutically effective amount of the essential oil according to claim 1.

15. The method of claim 14, wherein said essential oil is administered to a patient in need thereof by one or more of topical, rectal, intramuscular, systemic, intravenous, oral routes.

16. A method of treating melanoma in a patient in need thereof comprising administering to the patient a therapeutically effective amount of the essential oil according to claim 1 in combination with one or more anticancer drugs.

17. The method according to claim 16, wherein said essential oil and said one or more anticancer drugs are administered concomitantly or in sequence.

18. The method according to claim 16, wherein said anticancer drug is selected from anticancer MEK inhibitors, mutated or non-mutated BRAF inhibitors, c-KIT inhibitors, or mitotic inhibitors.

19. The method according to claim 18, wherein said anticancer MEK inhibitors are trametinib, cobimetinib, binimetinib, said anticancer mutated or non-mutated BRAF inhibitors are vemurafenib, dabrafenib, encorafenib, said c-KIT inhibitors are imatinib, nilotinib, said antimitotic inhibitors are mebendazol, vincristine, vinblastine, paclitaxel, or docetaxel.

20. The method according to claim 16, wherein said melanoma is cutaneous melanoma or metastatic melanoma.

21. The method according to claim 16, wherein said essential oil and said one or more anticancer are administered to a patient in need thereof by one or more of topical, rectal, intramuscular, systemic, intravenous, or oral routes.

22. The method according to claim 21, wherein said essential oil is administered topically and by one or more of rectal, intramuscular, systemic, intravenous, oral routes.

23. A pharmaceutical composition comprising the essential oil of Cinnamomum zeylanicum as defined in claim 1 and at least one pharmaceutically acceptable excipient.

24. The pharmaceutical composition according to claim 23, wherein said essential oil of Cinnamomum zeylanicum is present in an amount between 50 and 200 mg with respect to the total weight of the composition.
